(19) United States
(12) Patent Application Publication
(10) Pub. No.: US 2024/0248057 A1
(43) Pub. Date: Jul. 25, 2024

(54) METHOD FOR PRODUCING A SENSOR, AND SENSOR

(71) Applicant: AIT AUSTRIAN INSTITUTE OF TECHNOLOGY GMBH, Wein (AT)

(72) Inventors: Johannes BINTINGER, Tulln (AT); Patrik ASPERMAIR, Wein (AT); Ciril REINER-ROZMAN, Tulln (AT)

(73) Assignee: AIT AUSTRIAN INSTITUTE OF TECHNOLOGY GMBH, Wein (AT)

(21) Appl. No.: 18/559,892

(22) PCT Filed: May 10, 2022
(86) PCT No.: PCT/AT2022/060164

§ 371 Date: Nov. 9, 2023

(30) Foreign Application Priority Data
May 12, 2021 (AT) A 50363/2021

(51) Int. Cl. G01N 27/12; G01N 33/00

(52) U.S. Cl. CPC G01N 27/125; G01N 27/122; G01N 33/0027

(57) ABSTRACT

A method for producing a sensor comprising a circuit with multiple sensor substances. According to the invention, it is provided that electrical conductivities of sensor substances from a sensor substance list, which sensor substances each have an electrical conductivity dependent on a concentration of one or more gases in an environment of the sensor substance, are measured in environments with different concentrations of different gases and optionally different humidity levels and/or different temperatures, in order to determine sensitivities of the conductivities of the individual sensor substances for the different gases, wherein the measured conductivities and determined sensitivities of the sensor substances from the sensor substance list are stored in a database, whereupon, depending on requirements for the sensor with respect to gas concentrations in an environment which are to be ascertained using the sensor as well as on predefined usage conditions, in particular with respect to a humidity level and/or a temperature, at least two sensor substances from the sensor substance list are selected for the gas concentrations that are to be ascertained, based on the sensitivity stored in the database for the respective sensor substances, which selected sensor substances are integrated into an electrical circuit such that a conductivity of the sensor substances can be measured, so that the concentration of the gas constituent that is to be ascertained and/or a change in the concentration of the gas constituent that is to be ascertained in the environment of the circuit can be ascertained by measurement of the conductances of the sensor substances in the circuit and/or by measurement of changes in the conductances of the sensor substances in the circuit. Furthermore, a sensor for measuring gas constituents in the air of an environment is shown, comprising an electrical circuit which has at least two sensor substances from a sensor substance list, wherein conductivities of the sensor substances can be ascertained by means of the circuit. Additionally, a method for ascertaining gas constituents in an environment is proposed.

The invention relates to a method for producing a sensor comprising a circuit with multiple sensor substances.

The invention furthermore relates to a sensor for measuring gas constituents in the air of an environment, which sensor comprises an electrical circuit.

Sensors for ascertaining constituents of gases in the air of an environment have become known from the prior art. A disadvantage of such sensors is, however, that they can only be produced with great effort and therefore high costs.

Yet a need for cost-efficient sensors exists, in particular in order to be able to measure odors perceptible to humans in an automated and cost-efficient manner.

This is addressed by the invention. The object of the invention is to specify a method of the type named at the outset with which a particularly cost-efficient sensor for measuring gas constituents in the air of an environment can be produced.

A sensor of this type shall also be specified.

In addition, a method for ascertaining gas constituents in the air of an environment using a sensor system shall be specified, which method can be implemented with particular cost-efficiency.

The first object is attained according to the invention with a method of the type named at the outset, wherein electrical conductivities of sensor substances from a sensor substance list, which sensor substances each have an electrical conductivity dependent on a concentration of one or more gases in an environment of the sensor substance, are measured in environments having different concentrations of different gases and optionally different humidity levels and/or different temperatures, in order to determine sensitivities of the conductivities of the individual sensor substances for the different gases, wherein the measured conductivities and determined sensitivities of the sensor substances of the sensor substance list are stored in a database, whereupon, depending on requirements for the sensor with respect to gas concentrations in an environment which are to be ascertained using the sensor as well as on predefined usage conditions, in particular with respect to a humidity level and/or a temperature, at least two sensor substances from the sensor substance list are selected for the gas concentrations to be ascertained, based on the sensitivity stored in the database for the respective sensor substances, which selected sensor substances are integrated into an electrical circuit such that a conductivity of the sensor substances can be measured, so that the concentration of the gas constituent to be determined and/or a change in the concentration of the gas constituent to be determined in the environment of the circuit can be ascertained by measurement of the conductances of the sensor substances in the circuit and/or by measurement of changes in the conductances of the sensor substances in the circuit.

In the course of the invention, it was found that, depending on the requirements for a sensor with respect to gas constituents that are to be ascertained and to usage conditions, certain sensor substances exhibit a high specificity and, at the same time, a high selectivity with respect to the conductance, so that, depending on the usage conditions and the gas constituents that are to be ascertained, the conductances change for some sensor substances. Thus, by means of the change in the electrical conductances of the sensor substances, it is possible to deduce a specific concentration of a specific gas in an environment of the sensor that is to be produced, and/or to deduce a change in a concentration of a corresponding gas in an environment of the sensor via a change in the conductance.

Furthermore, a correlation of a change in a conductance of one sensor substance with a change in a conductance of another sensor substance can possibly be dependent on gas constituents in an environment, so that it is possible to deduce gas constituents on the basis of changes in conductance of individual sensor substances relative to one another.

In contrast to a gas chromatograph, for example, a sensor formed in a method according to the invention is thus typically only suitable for the measurement of specific gas constituents in an environment under specific usage conditions, but is also able to be produced at only a fraction of the cost of a corresponding gas chromatograph, so that sensors produced according to the invention are also suitable for low-cost applications, for example in order to measure the composition of waste in a refuse container. With a method according to the invention, it is thus possible to produce sensors at costs of less than one euro.

Furthermore, sensors produced according to the invention can also be miniaturized and operated in a very wide range of applied voltages, for example even at a voltage of only 1 mV, and can thus also be operated in a mobile manner by means of an energy storage device, in particular by means of an electric battery or a storage battery.

According to the invention, sensor substances that qualify for a corresponding sensor are thus analyzed, within the scope of upstream tests, with respect to whether and, where applicable, in what way the individual sensor substances have an electrical conductance dependent on a concentration of the most diverse gas constituents in an environment. Because the sensitivity of the conductance is thereby examined in relation to the most diverse gas constituents in an environment, there thus also results a specificity of a change in the conductance of the individual sensor substances for one or more gas constituents in an environment. The tests can be carried out at different humidity levels of an environment and at different temperatures, as well as with different exposure times. Furthermore, the tests can also be carried out with different states of the respective sensor material, for example with different states of the sensor material that result from different speeds at which the sensor materials are applied to a substrate. In addition, the tests can also be carried out at different points in time following an exchange of volume of a gas in an environment of the sensor.

A behavior of the sensor substances can also be dependent on a prior treatment of the sensor substances. For example, it can be possible for the behavior or the sensitivity and/or specificity for individual gas constituents to be influenced by a prior exposure to a particularly high or low temperature, an electromagnetic field, or other environmental parameters. This change in behavior can also be analyzed in tests and, depending on the requirements for the sensor, the sensor substances can be treated accordingly. Furthermore, in the evaluation of sensor signals, it is possible to take into consideration which environmental parameters these sensor substances were hitherto exposed to, possibly also during a usage in the sensor, so that measurement results can be adjusted accordingly.

For the purposes of this application, aerosols shall also be understood as gases.

The database can, for example, be saved and evaluated directly on the sensor, in a centralized cloud solution or a data storage device connected via a data connection, or decentrally by means of blockchain methods.

For this purpose, it can in particular also be provided that tests are carried out involving variation of the following parameters:
diameter of a nozzle with which the sensor substance is applied to a substrate;
distance of the nozzle from a substrate to which the sensor substance is applied using the nozzle;
temperature of the substrate to which the sensor substance is applied, in relation to a boiling temperature of a solvent with which the sensor substance is brought into a liquid state;
spacing of conductor paths;
modification of a sensor surface;
doping of sensor substances;
addition of thickening agents, in particular such as agar-agar or hypermellose, to the sensor substances;
surface modifications, and/or
cleaning steps.

By means of interpolation, it is possible to extrapolate intermediate values between the measurement results ascertained in tests.

The results of these trials are stored in a database, whereupon, depending on requirements for a sensor that is to be produced, the respective sensor substances are selected, based on the data stored in the database, which exhibit a beneficial selectivity, and possibly a beneficial specificity, for the gas constituents that are to be ascertained using the sensor and for the predefined usage conditions, after which the sensor materials are introduced accordingly into a circuit, so that, by means of the circuit, conductances of the sensor materials and changes in the conductances of the sensor materials can be measured in order to ascertain gas constituents in an environment with the aid of the conductances of the sensor materials using the sensor.

It can also be provided that data that are recorded by means of the sensors during a usage or in a field use, are continuously incorporated into a data collection, such as a cloud or a blockchain for example, and are used for the automated optimization of the selection process.

It can also be provided that sensor materials are selected which exhibit complementary sensitivities and selectivities with respect to the gas constituents that are to be analyzed.

It is beneficial if the conductivities of the sensor substances are measured with different gas concentrations, different humidity levels, and different temperatures. These data are then typically also stored in the database, so that, based on given usage conditions and gas constituents that are to be ascertained using the sensor, it is possible, with the aid of the database, to determine in a particularly accurate and efficient manner which sensor substances should be selected for the sensor.

It is preferably provided that the conductivities of the sensor substances are ascertained at different concentrations of one or more volatile organic gases in the atmosphere or an environment surrounding the sensor. For example, the conductivities can be ascertained at different concentrations of one or more of the following substances: 1,5-diaminopentane (C₅H₁₄N₂), butane-1,4-diamine (C₄H₁₂N₂), ethene (C₂H₄), triethylamine (C₆H₁₅N), methylsulfonylmethane (C₂H₆S), 1-methyl-4-(1-methylethenyl)cyclohexene (C₁₀H₁₆), methyl acetate (C₃H₆O₂), acetic acid (C₂H₄O₂), ethanol (C₂H₆O), 2-propanol (C₃H₈O), acetone (C₂H₆O).

It can also be provided that conductivities of at least one sensor substance are measured in different doping states, wherein a doped state of the sensor substance is formed, for example, by mixtures of the sensor substance, in particular a polymer, with a dopant, in particular F4TCNQ (2,3,5,6-tetrafluoro-7,7,8,8-tetracyanoquinodimethane). For example, 4-dodecylbenzenesulfonic acid and/or iron(III) p-toluenesulfonate can also be employed as dopants. It has thus been shown that doping states of sensor substances in particular have a significant influence on a behavior of the sensor substance, and therefore that different behaviors of the same sensor substance can be achieved merely by doping. For example, a conductance of the sensor substance PANI in an undoped state that is exposed to 2,3-butanedione differs markedly from an obtainable conductance of the same sensor substance in a doped state, which is achieved by mixture with 4.25 wt % F4TCNQ. Analogously, the behavior also changes markedly with respect to ethanol, for example, depending on whether PANI is employed in a doped or undoped state. Accordingly, sensors with different behavior can also be formed with identical sensor substances, but with different doping states. Correspondingly, it is beneficial if not only the sensor substances themselves, but also the doping states are saved in the database, in order to be able to select doped or undoped sensor substances for the sensor as needed.

Volatile substances in any desired aggregate states (for example, gases or primary or secondary aerosols), in particular organic gases, —VOC, short for volatile organic compounds—constitute organic compounds which are volatile at room temperature or transition to the gas phase at least at a reasonably moderate temperature, such as those organic compounds which occur, for example, in industrial processes, in the decomposition of refuse material, in the breath of humans, or the like. Within the scope of the invention, this includes in particular hydrocarbons with up to 20, in particular with up to approximately ten carbon atoms, wherein the carbon atoms can form branched or unbranched chains and/or can be present in a ring shape.

Compounds with one carbon atom, such as methane, CH₄, for example, are also subsumed therein within the scope of the invention. Ring-shaped carbon compounds with up to 20, preferably up to ten carbon atoms, are likewise included therein, for example aromatic compounds such as benzene, toluene, or saturated compounds such as cyclohexane. Both for chain-shaped molecules and also for ring-shaped molecules, one or more carbon atoms can be substituted, for example, by nitrogen (N) and/or oxygen (O) and/or sulfur (S), so that, according to the invention, volatile heterocyclic compounds are also included, for example pyridine. The volatile organic compounds also include hydrocarbons that are substituted, for example, with halogens such as fluorine (F), chlorine (Cl) and/or bromine (Br), thiols (—SH), hydroxyls (—OH), carboxylic acids (—COOH), and derivatives thereof, or the like. Also included are hydrocarbons with double bonds as well as alkenes such as ethene, or triple bonds, and therefore alkynes such as ethyne, for example.

Even though it is preferably volatile organic gases that are measured, gas compositions that cannot be counted among this category, such as carbon dioxide (CO₂), ammonia (NH₃), hydrogen (H₂), nitrogen (N₂), oxygen (O₂), and/or hydrogen sulfide (H₂S) for example, can also be analyzed with respect to components and concentrations thereof.

It is also possible, depending on the application case, to measure corresponding mixtures of volatile organic gases and inorganic gases such as nitrogen, oxygen, and hydrogen, as well as other components. One example of this is the measuring of biogas, which normally also contains nitrogen, oxygen, hydrogen sulfide, hydrogen, and ammonia in addition to methane and carbon dioxide as main constituents. This can also be applied to gases present or forming in a refuse container, which gases can also contain additional or different volatile organic gases. It is thus possible to ascertain a multi-component gas mixture having volatile organic gases and inorganic gases, such as carbon dioxide for example, possibly in a quasi-continuous manner, or possibly also in real-time or at least near real-time.

It is beneficial if the sensor substances are applied in a liquid state to a substrate, in particular to a printed circuit board that comprises electrical conductors, whereupon the sensor substances harden on the substrate, wherein the sensor substances connect electrical conductors so that an electrical conductance of the sensor substances can be ascertained via the electrical conductors of the substrate. The substrate itself is typically electrically non-conducting, or insulating, but can, as is typical for printed circuit boards, comprise electrical conductors by means of which a conductivity of the sensor substances can be measured. As a result, it is possible to form a particularly simple, cost-efficient sensor in an easy manner. The individual sensor substances can thereby be brought into a liquid state in the most diverse ways known from the prior art, for example by heating, by adding a solvent that remains in the sensor substance during a hardening or is removed, in particular vaporized or evaporated, out of said sensor substance.

Furthermore, individual sensor substances can also be brought into a liquid state through an application of mechanical force.

The substrate can be modified on the surface, in order to obtain a beneficial surface energy and wettability.

The sensor substances can, in principle, be applied in any desired manner, for example by pipetting or another type of application, in particular an automatic application. It is preferably provided that the sensor substances are printed onto the printed circuit board by means of a printer. As a result, a layer thickness of the sensor substances on the printed circuit board can also be defined with particular accuracy, which layer thickness can in turn be relevant for the conductance of the sensor substance on the printed circuit board or a sensitivity and specificity of the sensor substance for different gas constituents.

Furthermore, a polymerization can also first take place on the substrate or on electrodes of the circuit.

It is particularly beneficial if the sensor substances are dissolved in a solvent in order to bring the sensor substances into a liquid state. As a result, a liquid state that can be accurately defined is obtainable, in order to be able to apply the sensor substance to a printed circuit board or the like with precision in a repeatable process step, so that the sensor substances can be introduced into the circuit in a precisely reproducible manner.

During production of the sensor substances or integration of the sensor substances into the circuit, nucleation agents and/or thickening agents can also be employed, for example in order to expedite a crystallization in the case of a polymer.

Furthermore, thickening agents can also be employed to increase the viscosity in order to improve the targeted application to the sensor surface or the substrate.

It is advantageous if sensitivities of the conductances of the sensor substances for different gases in an environment are ascertained depending on one or more parameters, in particular printing parameters, with which the sensor substances are applied to a substrate, whereupon these dependencies are stored in the database, after which, based on data stored in the database and depending on requirements for the sensor with respect to gas concentrations in an environment that are ascertained using the sensor as well as on predefined usage conditions, in particular with respect to a humidity level and/or a temperature, parameters for the application of the individual sensor substances to the substrate are selected, whereupon the sensor substances are applied to the substrate using the corresponding parameters. As a result, a sensor can easily be formed which is optimally embodied for predefined usage conditions and for gas constituents that are to be ascertained.

It is preferably provided that sensitivities of the conductances of the sensor substances for different gases in an environment are ascertained depending on a speed at which the sensor substances are applied to a substrate, whereupon these dependencies are stored in the database, after which, based on the data stored in the databased and depending on requirements for the sensor with respect to gas concentrations in an environment that are to be ascertained using the sensor as well as on predefined usage conditions, in particular with respect to a humidity level and/or a temperature, speeds for the application of the individual sensor substances to the substrate are selected, whereupon the sensor substances are applied to the substrate at corresponding speeds, in particular at speeds of 0.2 μL/s to 30 μL/s.

Typically, different sensor substances are applied at different speeds, although, in principle, equal speeds can, of course, also be selected for two or more different sensor substances, depending on which application speed results in a beneficial behavior of the respective sensor substance, in particular in a high sensitivity and/or specificity with respect to specific gas constituents.

Alternatively or additionally to the application-speed printing parameter, the printing parameters of sensor substance temperature, substrate temperature, pressure, and the like can also be varied, and the effects on the conductance can be assessed, in order to accordingly obtain beneficial properties of the sensor substances through an application of corresponding printing parameters during the application of the sensor substances.

It has thus been shown that printing parameters have an influence on a sensor behavior, which is why the sensor substance or substances are typically applied at one or more speeds which are selected depending on which behavior of the individual sensor substances is desired or which gas constituents are to be ascertainable using the sensor. Thus, it has been shown that different printing speeds, for example of 1 mm/s, 0.4 mm/s, and 0.1 mm/s, result in different material structures, possibly crystal structures, of the hardened sensor substances, in particular of polymers and low-molecular-weight compounds, which different material structures lead to different conductances of the sensor substances under identical environmental conditions, or to increased and/or decreased sensitivities and/or specificities with respect to individual gas constituents. In other words, in the case of identical sensor substances, a sensor behavior can be influenced by different printing parameters.

Thus, through a corresponding application of the sensor substances to the substrate, which normally comprises electrical conductors in accordance with a circuit, the sensor substances are arranged in the circuit. In a particular simple embodiment, the circuit can be formed by two electrical conductors that are connected by one or more sensor substances, so that conductances and changes in the conductances of the sensor substances can be measured via the electrical conductors.

In order to be able to form a sensor with which gas constituents in an environment can be ascertained with particular precision, it is preferably provided that the liquid sensor substances are applied to the substrate using a nozzle that is moved relative to the substrate, wherein the nozzle preferably has a nozzle diameter of less than 1 mm, in particular 100 μm to 500 μm.

It has been shown that it is beneficial if the sensor substances are applied to the substrate at a speed of 0.1 mm/s to 10 mm/s, in particular 0.2 mm/s to 4 mm/s, in particular using a nozzle as described above. As a result, a layer of the sensor material on the substrate with behavior that can be reproduced with particular accuracy is obtained.

Particularly if the sensor substance is brought into a liquid state through the addition of a solvent, which solvent is vaporized or evaporated after application of the sensor substance to the substrate, it is beneficial if the substrate is kept at a predefined temperature during the application of the sensor substances, in particular at a temperature of 25° C. to 120° C. Specifically, a reliable vaporization of the solvent can then be ensured, whereby microscopic cavities can be generated in particular which can enlarge an active surface and improve a sensitivity and/or specificity. In addition, a predefined temperature of the substrate during application of the sensor substances is also beneficial in order to be able to produce reproducible sensors having an essentially identical behavior.

It has been shown that, in the case of certain sensor substances, certain temperatures during application have an influence on a sensitivity and/or specificity of the respective sensor substance on the sensor, so that a characteristic of the sensor can be influenced by the temperature during application of the sensor substance, possibly in combination with other application parameters such as a speed, for example. Typically, this temperature is maintained until the sensor substance hardens or dries.

It is particularly beneficial if the substrate is positioned on a heating device, in particular a hot plate, which is kept at a constant temperature, in particular a temperature at which a solvent with which the sensor substance is brought into a liquid state vaporizes. Using a corresponding heating device, a temperature of the substrate can be set with particular accuracy, so that conditions under which the sensor substances are applied to the substrate and introduced into the circuit can be specified with particular exactness. This makes it possible to obtain with particular ease sensors having predictable behavior.

During application of the sensor substances, the substrate typically has a temperature of 15° C. to 150° C., preferably 30° C. to 100° C., in particular 40° C. to 80° C.

For a sensor produced in a method according to the invention, any desired sensor substances that exhibit an electrical conductance dependent on a gas in an environment can, in principle, be employed. It has proven particularly effective if at least one of the sensor substances that are integrated into the circuit comprises a polymer. Polymers are, on the one hand, affordable to produce and, on the other hand, have advantageous chemical properties which qualify them for use on a corresponding sensor. This applies in particular to organic polymers and preferably, for the purposes of the invention, to electrically conductive organic polymers.

The polymers can also be embodied as doped polymers, for example mixtures of polymers with molecular dopants, in particular F4TCNQ (2,3,5,6-tetrafluoro-7,7,8,8-tetracyanoquinodimethane), wherein 4-dodecylbenzenesulfonic acid and/or iron(III) p-toluenesulfonate can be employed as dopants, for example.

Furthermore, it can be provided that the sensor substances comprise metal oxides, in particular copper oxide or zinc oxide, metal particles, in particular gold particles, and/or polymers, in particular organic, preferably electrically conductive polymers, preferably electrically conductive, in particular organic electrically conductive polymers, or are composed thereof, especially since said substances also have a conductance dependent on gas concentrations in an environment.

It has been shown that metal oxides as sensor substances are particularly well-suited for operating temperatures of 250° C. to 450° C., whereas polymers as sensor substances in particular are especially well-suited for sensors that are operated at room temperature. In order to obtain a beneficial sensor behavior, however, a heating and/or cooling device can also be provided with which a beneficial temperature of the sensor substance is achieved. Accordingly, the entire sensor or only one or more sensor substances can be brought to a predefined temperature in a targeted manner. In order to achieve a particularly low energy requirement of the sensor, such sensor substances as can be operated well in the corresponding temperature range are preferably employed. Thus, for sensors that are to be operated at room temperature, polymers are preferably employed as sensor substances.

For example, it can be provided that the sensor substances contain Mn₃O₄. ZrO₂, TiO₂, CeO₂, ZrO₂, ZnO, TiO₂, Cr₂O₃, Co₃O₄ and/or SnO₂ or are composed of one or more of said metal oxides.

In principle, those substances which exhibit a sufficient selectivity and sensitivity for the respective gas that is to be ascertained, under consideration of desired detection limits, qualify as sensor substances. It is not precluded that, with respect to a single gas constituent that is to be ascertained, for example a specific volatile organic gas, two or more sensor substances are employed, for example in order to be able to cover different concentration ranges and/or to statistically better support the measurement result.

It is particularly beneficial if the sensor substance list contains polymers, preferably conductive, in particular organic electrically conductive polymers, and/or metal oxides, in particular copper oxide or zinc oxide, and/or metal particles, in particular gold particles, or is composed of substances of this type.

In a first embodiment, the sensor substance can also contain nanoparticles, such as those which are already known per se from the prior art. For example, these can be nanoparticles of gold or platinum that react to certain gases.

Another class of materials that can be used for the sensor substances is metal oxides such as copper oxide or zinc oxide, for example. Metal oxides, similarly to metal particles, can be employed particularly if the metal oxides exhibit a certain oxidation resistance, such as gold particles for example. The metal oxides can thereby be present as bulk material or, similarly to the noted nanoparticles, as nanomaterials, for example as metal oxide nanowires with a length of up to maximally 500 nanometers, for example, and a width of no more than 50 nanometers, for example.

It is particularly preferred if polymers, in particular organic polymers, are employed as sensor substances, since these can be relatively easily printed, and can thus be applied to virtually any desired substrate from a printable mass.

Among the usable polymers, electrically conductive polymers in particular have proven to be especially well-suited sensor substances. The electrically conductive polymers, typically organic polymers, can, like other polymers, also in turn be present as nanowires, though this is not imperative. The polymers can also be applied as a planar substance to a substrate.

The electrically conductive polymers can be connected to other sensor substances on a sensor. However, if electrically conductive polymers are employed, they are preferably employed as exclusive sensor substances, since in this case all sensor substances can be applied as a planar coating layer with a thickness of less than one millimeter, preferably less than 0.5 millimeters, under largely identical conditions, for example by printing, in particular inkjet printing, or in a different manner such as spraying, spin coating, dip coating or other planar coating methods.

Suitable additives can also be admixed in the process, for example carbon black or soot, in order to adapt the conductivity. The term “exclusive sensor substances” thus also includes suitable auxiliary materials which, however, are not more than 20 percent by weight (wt %), preferably not more than 10 wt %, in particular not more than 5 wt %, in relation to the entire mass (polymer and auxiliary materials).

In particular, polyurethane, polyaniline (PANI) and derivatives thereof, possibly also in mixtures with carbon black, poly(3-hexylthiophene-2,5-diyl) [P3HT] and derivatives thereof, poly(3,4-ethylenedioxythiophene):polystyrene sulfonate (PEDOT:PSS), poly {2,2′-[(2,5-bis(2-hexyldecyl)-3,6-dioxo-2,3,5,6-tetrahydropyrrolo[3,4-c]pyrrole-1,4-diyl)dithiophene]-5,5′-diyl-alt-thiophen-2,5-diyl)} [PDPP3T], polyepichlorohydrin (PECH) or poly[(chloromethyl)ethylene oxide], poly[2-(3-thienyl)ethoxy-4-butylsulfonate], and other polymers, including copolymers, can be used as electrically conductive polymers.

Other examples of substances which the sensor substance list can comprise or of which the sensor substance list can be composed, can be seen from Table 1 below, together with the structural forms and CAS no.

TABLE 1

| Substance | Chemical Name | CAS No. | Illustration |
|---|---|---|---|
| Polyaniline | | | |
| nanowires | | | |
| & NP | | | |
| Polyaniline (PANI) | Polyaniline | 25233- 30-1 | 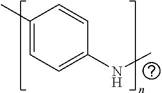 |
| PANI/carbon | | | |
| black | | | |
| P3HT | Poly(3- hexylthiophene-2,5- diyl) | 104934- 50-1 | 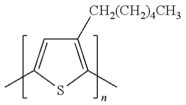 |
| PEDOT:PSS | Poly(3,4- ethylenedioxythio- phene) poly(styrene- sulfonate) | 155090- 83-8 | 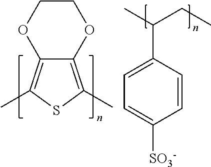 |
| PDPP3T | Poly{2,2′-[(2,5- bis(2-hexyldecyl)- 3,6-dioxo-2,3,5,6- tetrahydropyrrolo [3,4-c]pyrrole-1,4- diyl)dithiophene]- 5,5′-diyl-alt- thiophen-2,5-diyl} | 1198291- 01-8 | 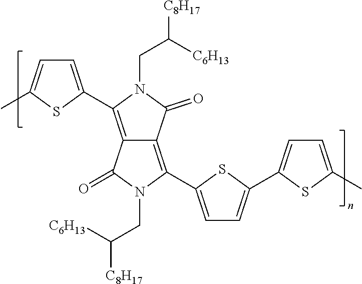 |
| Polyepichloro- hydrin (PECH) | Poly[(chloromethyl) ethylene oxide] | 24969- 06-0 | 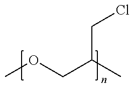 |
| Poly(ethylene- vinyl acetate) (PEVA) | Poly(ethylene-co- vinyl acetate) | 24937- 78-8 | 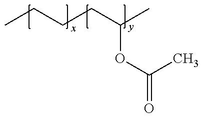 |
| PEVA | | | |
| TFB | Poly[(9,9- dioctylfluorenyl- 2,7-diyl)-co-(4,4′- (N-(4-sec- butylphenyl) diphenylamine)] | 220797- 16-0 | 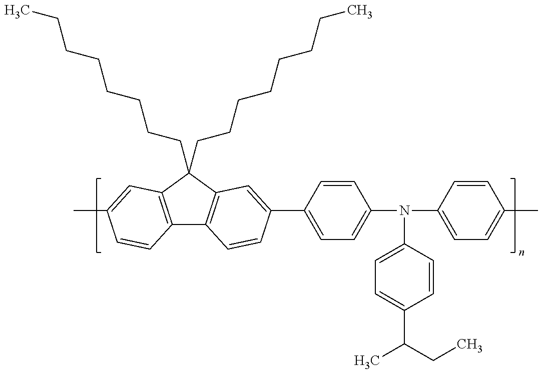 |
| Poly-TPD | 4-Butyl-N,N- diphenylaniline | 472960- 35-3 | 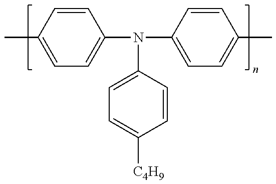 |
| HAT7 | 2,3,6,7,10,11- hexakis(heptyloxy) triphenylene | 69079- 52-3 | 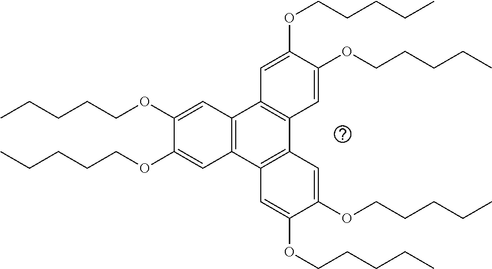 [SMILES: CCCCCOc1cc2c3cc(OCCCCC)c(OCCCCC)cc3c3cc(OCCCCC)c(OCCCCC)cc3c2cc1OCCCCC] |
| PBTTT | Poly[2,5-bis(3- tetradecylthiophen- 2-yl)thieno[3,2- b]thiophene] | 888491- 19-8 | 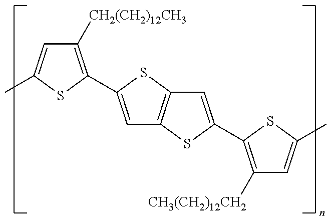 |
| F8BT | Poly(9,9- dioctylfluorene-alt- benzothiadiazole) | 210347- 52-7 | 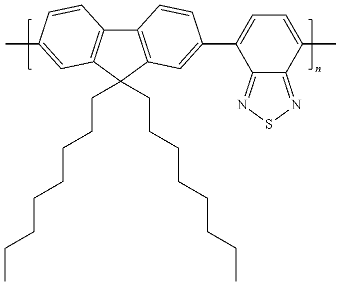 |
| PQT-12 | Poly(3,3′″- didodecyl [2,2′:5′,2″:5″,2′′′- quaterthiophene]- 5,5″-diyl), poly(4,4″- didodecyl [2,2′:5′,2″:5″,2′′′- quaterthiophene]- 5,5″′-diyl) | 827343- 06-6 | 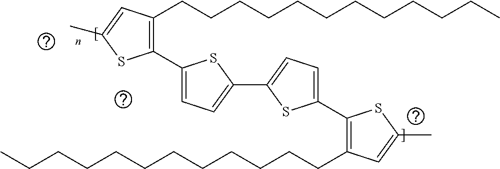 [SMILES: CCCCCCCCCCCCc1cc(C)sc1-c1ccc(-c2ccc(-c3sc(C)cc3CCCCCCCCCCCC)s2)s1] |
| | Poly[3-(potassium- 4- butanoate) thiophene- 2,5-diyl], regioregular | | 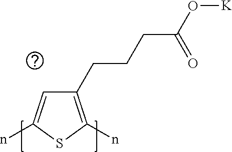 |
| | Poly[3-(potassium- 5-pentanoate) thiophene- 2,5-diyl], regioregular | | 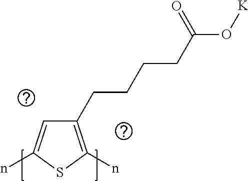 |
| | Poly[3-(potassium- 6-hexanoate) thiophene- 2,5-diyl], regioregular | | 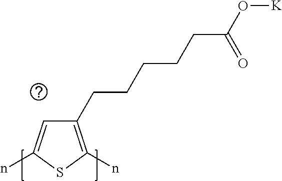 |
| PIB | Poly(2-methylprop- 1-ene) | 9003-27-4 | 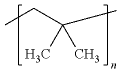 |
| Polypyrrole (Ppy) | Poly(1H-pyrrole) | 30604- 81-0 | 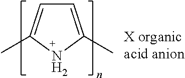 |
| Polybutadiene (PBD) | Poly(buta-1,3- diene) | 9003-17-2 | 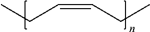 |
| PNVP | N-Vinyl-2- pyrrolidone | 9003-39-8 | 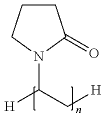 |
| PBDB-T (PCE12) | Poly[(2,6-(4,8- bis(5-(2- ethylhexyl) thiophen- 2-yl)-benzo[1,2- b:4,5-b′] dithiophene))-alt- (5,5-(1′,3′-di-2- thienyl-5′,7′-bis(2- ethylhexyl)benzo [1′,2′-c:4′,5′- c′]dithiophene-4,8- dione)] | 1415929- 80-4 | 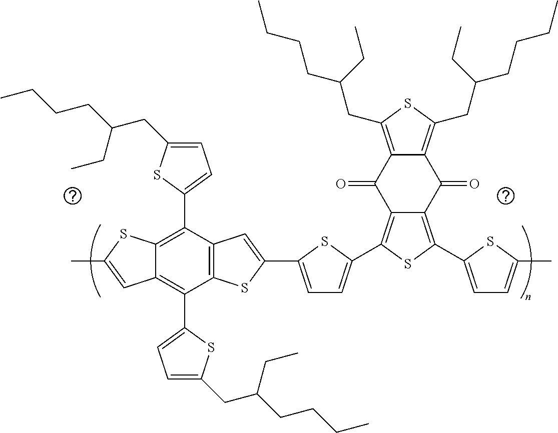 |
| PNDI (2OD)2T | Poly{[N,N′-bis(2- octyldodecyl)- naphthalene-1,4,5,8- bis(dicarboximide)- 2,6-diyl]-alt-5,5′- (2,2′-bithiophene)} | 1100243- 40-0 | 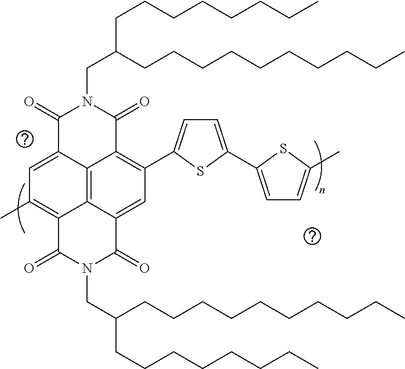 |
| DPP-DTT | Poly[[2,3,5,6- tetrahydro-2,5- bis(2- octyldodecyl)- 3,6- dioxopyrrolo[3,4- c]pyrrole-1,4-diyl]- 2,5-thio- phenediylthieno [3,2-b]thiophene- 2,5-diyl-2,5- thiophenediyl] | 1260685- 66-2 (1444870- 74-9) | 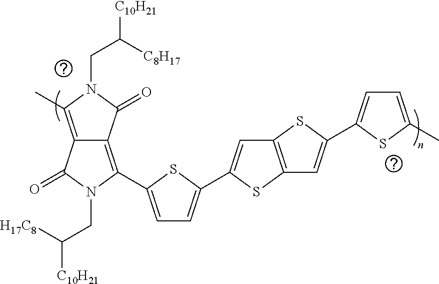 |
| PTEBS | Sodium poly[2-(3- thienyl)ethoxy-4- butylsulfonate] | | 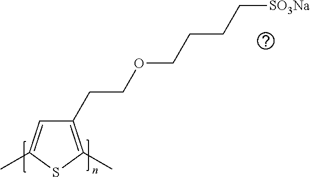 |
| PBDTT-DPP | Poly{2,6′-4,8-di(5- ethylhexylthienyl) benzo[1,2-b;3,4- b]dithiophene-alt- 5,5′-dibutyloctyl- 3,6-bis(5-thiophen- 2-yl)pyrrolo[3,4- c]pyrrole-1,4- dione} | 1380582- 98-8 | 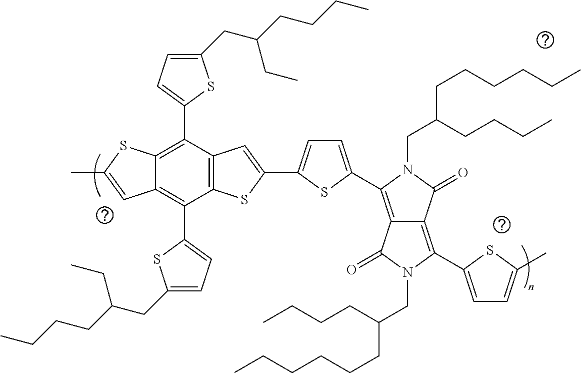 |
| PSBTBT | Poly[(4,4-bis(2- ethylhexyl)- dithieno[3,2- b:2′,3′- d]silole)-2,6-diyl- alt-](2,1,3- benzothiadiazole)- 4,7-diyl] | 1089687- 02-4 | 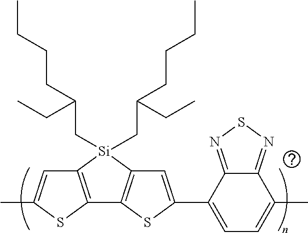 |
| C8-BTBT | 2,7-Dioctyl[1] benzothieno [3,2-b] [1]benzothiophene | 583050- 70-8 | 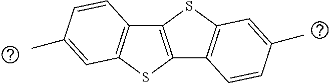 |
| Triphenyl- amine (TPA) | N,N- Diphenylaniline | 603-34-9 | 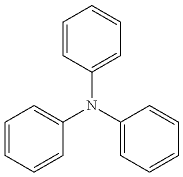 [SMILES: c1ccc(N(c2ccccc2)c2ccccc2)cc1] |
| PCDTBT | Poly[N-9′- heptadecanyl-2,7- carbazole-alt-5,5- (4′,7′-di-2-thienyl- 2′,1′,3′- benzothiadiazole)] | 958261- 50-2 | 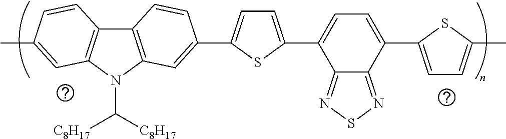 |
| PCP-Na | Chemical formula: (C₂₇H₂₂O₆S₄Na₂)ₙ | | 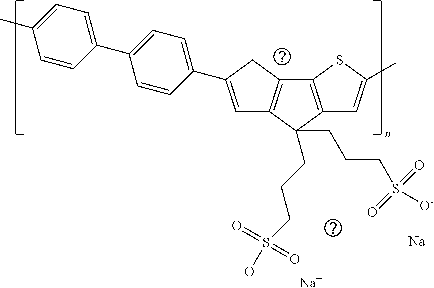 |

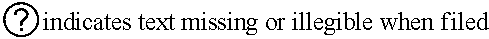

Those substances for which no CAS no. is stated can, as of the application date, be readily obtained from one of the internet sites rickemetals.com or brilliantmatters.com, for example, by providing the chemical name.

In a further development of the invention, it is possible that the sensor substances are aligned with the use in targeted manner. For example, the sensor substances for an establishing of volatile substances in refuse containers, and therefore possible conclusions about contents of a refuse container, can be composed according to Table 2 below, which also indicates which odors the respective substances are particularly sensitive to.

TABLE 2

| Chemical Structure | Name | Reference | Odor |
|---|---|---|---|
| 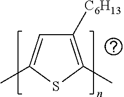 | P3HT | Chang, et al. J. Appl. Phys. 2006, 100, 014506 | Fish |
| 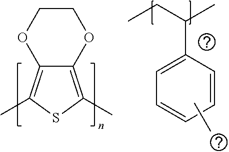 | PEDOT: PSS | Procedia Engineering 168, 2016, 1184-1187 | Vinegar |
| 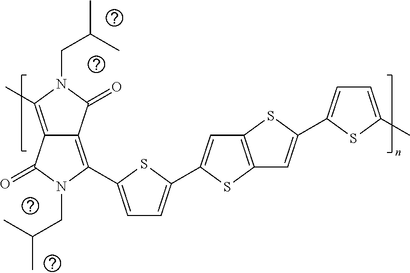 | DPP- DTT | J. Mater. Chem. C, 2019, 7, 1111-1130 | Lemon oil, orange oil |
| 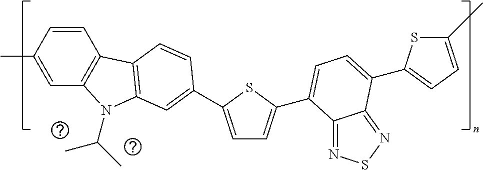 | PCDTBT | Sensors and Actuators B, 239, 2017, 734- 745 | Bleach |
| 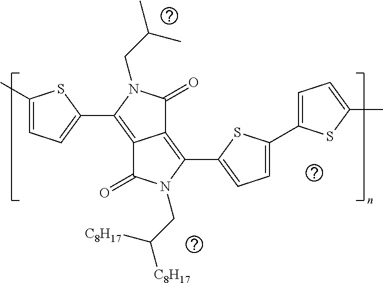 | PDPP3T | Adv. Sci., 2017, 4, 1700048 | Ethanol |
| 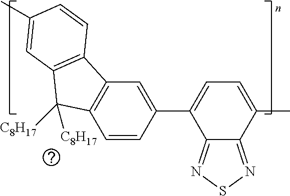 | F8BT | Mine Action, 2015, 201-4 | Moisture (water) |
| 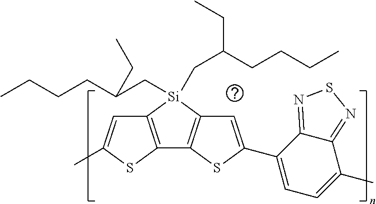 | PSBTBT | Adv. Funct. Mater., 2014, 24, 2240-2247 | Vinegar, fish |
| 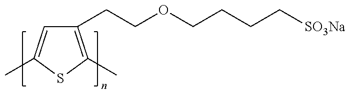 | PTEBS | Mater. Today, 2016, 19-9 | Vinegar, meat |

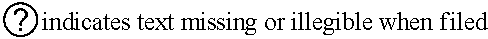

In a further development of the use of electrically conductive polymers as sensor substances, it can be provided that the electrically conductive polymers are modified. This includes in particular a modification of the electrically conductive polymers with peptide side chains. The peptide side chains employed can in this case be based on biopeptides, which, similarly to the human sense of smell, are sensitive to certain molecules. Thus, as a result of different analyte-polymer interactions based on aromatic and dipole-dipole interactions and hydrogen bonds, it is possible to control or adjust a sensitivity in a targeted manner via amino acids.

Different synthesis routes are open for the production of correspondingly functionalized electrically conductive polymers that can also be designated as biomimetic electrically conductive polymers. In a first strategy, 1,4-di-2-dienyl-1,4-butanedione can be reacted in a Paal-Knorr condensation reaction with an amino group of the selected amino acid sequence to produce the correspondingly substituted 2,5-di(2-dienyl)-pyrrole, which can then be electrochemically polymerized to produce poly(2,5-dienylpyrrole). Alternatively, by introducing an azide into the base pyrrole framework, it is possible to introduce an alkyne-substituted amino acid sequence using click chemistry (copper-catalyzed azide-alkyne cycloaddition). Thus, in both strategies, reactive bonds for coupling with a peptide sequence are subsequently available.

Graphene and in particular graphene compounds are also suitable as a sensor substance. Similarly to the case of electrically conductive polymers, graphene can in this case be combined by addition reactions with molecules that have a specificity for certain molecules. It is thereby possible, where necessary, to again resort to peptides or amino acid sequences that are coupled by reaction to spacer molecules which are linked to graphene as an electrically conductive base.

In this case, 1-pyrenebutyric acid or 1-pyrenebutyric acid N-hydroxysuccinimide ester can be used as a linker, for example. Because of x-x interactions, a pyrene group adheres to other conjugated double bonds (graphene, reduced graphene oxide and carbon nanotubes), wherein the carboxylic acid at the other end has the functionality necessary to link additional functionalities.

It is particularly beneficial if the sensor substances are selected based on substances noted in the following list:
PANI;
P3HT;
DPP-DTT;
PDPP3T;
PEDOT:PSS
F8BT;
PCDTBT;
PTEBS;
PSBTBT;
PCE-12;
C8-BTBT;
PCP-Na.

The sensor substances can thereby comprise one or more of the noted substances, or can be formed from one or more of the noted substances.

Here, “based on substances noted in the list” shall be understood as meaning that suitable derivatives of the substances noted in the list can also be selected.

It is preferably provided that, following integration of the sensor substances into the circuit, the sensor is calibrated in that the conductances of the individual sensor substances of the sensor are measured depending on the individual gas concentrations in an environment that are to be ascertained, the gas concentrations present in the environment during the calibration as well as a temperature and a relative humidity of the air being known, wherein the conductances of the sensor substances of the sensor are stored.

It is thus possible, based on actual concentrations of specific gas constituents in the environment, to determine a behavior of the sensor in order to deduce concentrations of gas constituents in an environment of the sensor for a later use of the sensor with the aid of the conductances of the individual sensor substances that are integrated into the sensor or into the circuit.

It is preferably provided that the conductances of the individual sensor substances of the sensor are measured at different temperatures, different humidity levels, and different exposure times, in order to determine influences of moisture and temperature on a sensor behavior. A calibration of the sensor can then take place with the aid of conductivities of the individual sensor substances depending on the gas mixtures in an environment surrounding the sensor, wherein, within the scope of pattern detection methods known from the prior art (pattern recognition and/or artificial neural networks), data can be used during operation of the sensor in order to deduce gases present in the environment by means of conductances of the individual sensor substances. Thus, through a combination of different sensor substances, the sensor can be employed for the targeted ascertaining of individual gas constituents, even though the individual sensor substances do not have an adequately high specificity for the respective gas.

It can furthermore be provided that the conductances of the individual sensor substances are measured at different ambient pressures, in particular at a pressure range of 0.3 bar to 4 bar, in order to also be able to account for these parameters or the ambient pressure to which the sensor is to be exposed during operation, during production of the sensor and in particular when selecting corresponding sensor substances.

It is beneficial if the conductances of the individual sensor substances of the sensor are ascertained in the presence of multiple different gases in an environment of the sensor.

The other object of the invention is attained according to the invention by a sensor of the type named at the outset which comprises at least two sensor substances from a sensor substance list, wherein conductivities of the sensor substances can be ascertained by means of the circuit. The sensor is preferably produced in a method according to the invention.

A corresponding sensor can be produced with little effort and therefore at low cost, and yet is suitable for ascertaining the presence, concentration, and/or change in a concentration of a gas constituent in an environment surrounding the sensor. Therefore, a corresponding sensor can, in particular, be employed for use in ascertaining a constituent of waste in a smart refuse container.

It is beneficial if at least six, preferably at least twelve, different sensor substances are arranged on the circuit, wherein the conductance of each of said sensor substances can be ascertained.

As a result, a comparatively high accuracy of the sensor can be achieved via an appropriate combination of different sensor substances, despite the inexpensive production process. For this purpose, such sensor substances as exhibit a particularly high sensitivity, selectivity, and/or a particularly high specificity for the gas constituent(s) that is/are to be ascertained are preferably combined and arranged on the circuit, in order to be able to accurately identify the gases that are to be ascertained, by a combination of the respective measured values with respect to the conductivity of the individual sensor substances.

One or more sensor substances that show no reaction to a gas constituent that is to be ascertained can also be employed in order to thus rule out the presence of other gases to which said sensor substance is sensitive, and to thus specifically verify the reactions of the other sensor substances integrated into the circuit. Such sensor substances are thus used for the negative control of specific gas constituents.

It is preferably provided that at least one of the sensor substances comprises a polymer or is composed of a polymer.

Furthermore, it can advantageously be provided that one or more of the sensor substances arranged in the circuit comprise metal oxides, in particular copper oxide or zinc oxide, metal particles, in particular gold particles, and/or polymers, in particular organic, preferably electrically conductive polymers, or are composed thereof.

It is advantageously provided that the sensor substance list is created based on the substances stated below, on the substances noted in Table 1 and/or the substances stated in Table 2, and/or is composed of said substances:
PANI;
P3HT;
DPP-DTT;
PDPP3T;
PEDOT:PSS;
F8BT;
PCDTBT;
PTEBS;
PSBTBT.

In this case, the sensor substance list can, of course, also comprise suitable derivatives of the noted substances. Suitable derivatives are those which still exhibit a sensitivity sufficient for the purpose of the invention.

The sensor substances can, in principle, be integrated into the circuit in the most diverse ways, so that a conductance of the sensor substance can be ascertained by means of the circuit. It is particularly preferred if it is provided that the sensor substances are printed onto the circuit.

Electrodes that are connected by the sensor substances are typically arranged on the circuit. As a result, it is possible to directly deduce a conductance of the sensor substance with the aid of an electrical conductance between the electrodes or an electrical resistance between the electrodes.

In order to be able to minimize or completely eliminate with particular ease measurement inaccuracies specifically due to aging effects, it is preferably provided that, in parallel and/or in series with each sensor substance that connects two electrodes of the circuit and is connected to an environment, an identical sensor substance is arranged in the circuit, which identical sensor substance is hermetically separated, or essentially hermetically separated, from the environment. Thus, to ascertain a gas constituent in an environment using a bridge circuit, a voltage divider, or the like, for example, a difference between the conductance of the sensor substance connected to the environment and the identical sensor substance hermetically separated from the environment can then be determined. As a result, effects of aging processes of the sensor substances and influences of temperature changes on the conductivity of the respective sensor substance are, for example, equalized during the measurement of gas constituents.

In particular, a differential measurement can thus be realized in this manner, by means of which measurement, only differences between the conductances of the individual sensor substances are registered, but aging effects which appear synchronously in multiple sensor substances are not taken into account.

For the measurement of a change in conductance, a voltage at a voltage divider formed by correspondingly arranged sensor substances is preferably measured by means of an analog-to-digital converter, and a measured value thus digitalized is further processed in a data processing device. With the voltage divider, a difference between the conductances of the sensor substances and a change in a conductance of a sensor substance relative to the conductance of a second sensor substance of the voltage divider can thus easily be measured.

A sensor according to the invention is typically used in combination with a data processing system, in order to be able to deduce gas constituents in an environment with the aid of measured conductances of the individual sensor substances, for example using calculated correlations and/or targeted utilization of multi-variant aspects of the individual sensor substances. Thus, the individual sensor substances can, for example, comprise different functional groups, in order that the overlapping specificities resulting therefrom are able to cover a largest possible range of analytes. In this case, for example, sensor substances which comprise a polymer can be combined on a sensor with sensor substances that comprise a metal oxide. Machine learning, pattern detection methods such as support vector machines in particular and artificial neural networks, supervised and unsupervised machine learning, and methods from probability calculation such as Gaussian mixture models, for example, can also be employed for evaluation. The sensor is thus typically employed as part of a sensor system which, in addition to the sensor, also comprises a data processing device.

It is therefore preferred that, in the case of a sensor system that comprises a sensor and a data processing device connected to the sensor, which data processing device in particular comprises a microprocessor and a data storage device, the sensor is embodied according to the invention.

The data processing device can thereby be employed for the most diverse fields of application, while the sensor can comprise different sensor substances depending on the area of application. It is therefore beneficial if the sensor can be detachably connected to the data processing device. A mechanical and electrical connection of the sensor to the data processing device can thereby take place in a force fit and/or form fit, for example.

It is particularly preferred if it is provided that the sensor is embodied as a plug-in card and is connected to the data processing device via a slot, so that the sensor can be plugged into the slot, wherein an electrical connection between the sensor and the data processing device can be produced by plugging the sensor into the slot. On the one hand, this enables a simple interchangeability of sensors, so that the sensor system can easily be adapted to different fields of application. In addition, due to an interchangeability of the sensor resulting from a detachable connection such as a plug connection, a potentially short service life of individual sensor substances can be cost-efficiently compensated without replacing the entire sensor system.

Furthermore, it is beneficial if the data processing device is suitable for performing a method wherein, with the aid of measured conductances of the sensor substances as well as sensitivities stored in a database for the individual sensor substances, concentrations of one or more gases in an environment of the sensor are ascertained. By means of the established sensitivities, a comparatively accurate ascertaining of gas constituents in an environment surrounding the sensor can then take place despite the cost-efficient sensor construction.

The database can be stored centrally or decentrally, for example also such that it is distributed on different data storage devices that are connected via an Internet of Things network.

In particular, it can be provided that a data processing software is designed such that, using said software, corresponding pathways are specifically assigned for different combinations of sensor substances, and a synchronization with the database with respect to an exact positioning of the sensor substances is carried out.

The data processing device can thereby also be employed to carry out pattern detection methods and methods which are known from an area of machine learning, in order to deduce gas constituents present in the environment with the aid of measured conductances of the individual sensor substances.

It can thereby also be beneficial if the sensor system comprises an acoustic sensor and/or an optical sensor, in particular a light sensor or a brightness sensor, in order to perform a measurement by means of the sensor, in particular such that said measurement is triggered by acoustic and/or optical changes in an environment of the sensor system. For example, a measurement can then be performed such that it is triggered by an opening sound and/or an abruptly-altered incidence of light. Specifically, the ascertaining of a composition of contents of refuse containers is thus possible in a particularly efficient manner. A measurement thus need not be performed continuously; rather, it is often sufficient to perform a measurement following a trigger event such as an opening of the refuse container.

Data of the trigger event can also be used, in particular by a software in the data processing device, to calculate probabilities of gas constituents that are to be ascertained. For example, if the sensor is used in a refuse container, a specific sound can imply that a beverage can has been thrown into said refuse container, so that there is a heightened probability of an increase in concentration of gas constituents in the refuse container, which gas constituents are associated with a beverage can. In the evaluation of the sensor signals or an automated interpretation of changes in conductance of the sensor substances, this heightened probability can be used in order to achieve a particularly high measurement accuracy through the combination of the data of the trigger event with the measured conductances of the sensor substances or the measurement of changes in the conductances.

Alternatively or additionally, other sensors can, of course, also be provided on the sensor system, for example distance sensors and/or motion or acceleration sensors.

In order to be able to ascertain changes in a gas composition in an environment of the sensor with particular rapidity, it can be envisaged that a transport device is provided with which a gas that is to be analyzed can be actively transported to the sensor substances of the sensor, in particular a pump, a compressor, a blower, or the like. A transport device of this type can, for example, be activated such that it is triggered by a trigger event, which trigger event can in turn be captured in an automated manner, for example by means of a distance sensor, a motion sensor, an acoustic sensor, and/or an optical sensor such as a camera, for example. The analysis can also include a headspace sampling method.

Furthermore, it can be provided that upstream of the sensor a volume, in particular an enclosed space which is fed by a transport device with a gas that is to be analyzed, is installed, in which volume a concentration of an analyte takes place.

An algorithm which can be stored in a program in the data processing device can then, for example, be designed such that an evaluation is carried out only for a predefined duration following the trigger event, for example an opening of a door or a lid. This can be expedient particularly if changes in a gas composition are essentially only expected when said trigger event occurs.

The further object is attained according to the invention by a method for ascertaining gas constituents in an environment using a sensor system, in which method a sensor system according to the invention is employed. The sensor system can thus be produced in a particularly cost-efficient manner, for which reason the method can also be implemented in a simple and cost-efficient manner.

It is preferably provided that, with the aid of measured conductances of the sensor substances and sensitivities stored in a database for the individual sensor substances, concentrations of one or more gases in an environment of the sensor are ascertained.

The method can be employed for the most diverse purposes, for example in order to monitor a smell in a sanitary installation in an automated manner to identify anomalies, in particular leaks. Furthermore, it can be provided that the method is employed to ascertain substances contained in a refuse container. Additional data that can be acquired using other sensors, for example a camera, can then also be incorporated into the method, for example in order to achieve a constant improvement of method results by machine learning and/or using methods of pattern detection.

In order to achieve a particularly high energy efficiency, it can be provided that a trigger event is captured and an analysis of gas constituents is carried out starting with the trigger event.

Thus, a continuous analysis of the gas constituents does not normally take place; rather, such an analysis occurs only when it has been triggered by a trigger event, which can also be captured in an automated manner, for example using an acoustic sensor, an optical sensor, a distance sensor, or the like. Thus, when the method is used on a refuse container, for example, an analysis can be performed whenever an opening and/or closing of a lid of the refuse container has been detected, especially since a composition of the waste in the refuse container typically only changes after such an event.

It can furthermore be provided that concentrations and/or changes of gas constituents in an environment of the sensor are ascertained on the basis of chronological changes in the conductances of the individual sensor substances that are measured starting with the trigger event.

It has thus been shown that different sensor substances react with different intensities to different gas constituents in an environment. Accordingly, not only is it possible to deduce a concentration and/or a change in a concentration of a gas constituent in an environment via a conductance, but also via a chronological change in the conductance, in particular following a trigger event.

Of course, an analysis can also take place in a time-dependent manner, for example a composition of a gas performed after fixed intervals of time. It can thereby also be provided that the intervals of time can be adjusted. Thus, even in the case of a continuous measurement, data points can be recorded at a chronological interval of only a few milliseconds or an interval of multiple minutes, for example. This optimizes an energy efficiency depending on particular requirements or a query that needs to be answered.

For example, with one sensor substance, two different gas constituents can result in the same conductance, yet the change in conductance can occur with different rapidity. Accordingly, by measuring chronological changes in the conductances of the individual sensor substances, it is possible to more accurately distinguish between different gas constituents without needing to arrange additional sensor substances on the sensor.

Additional features, advantages, and effects of the invention follow from the exemplary embodiment described below. In the drawings which are thereby referenced:

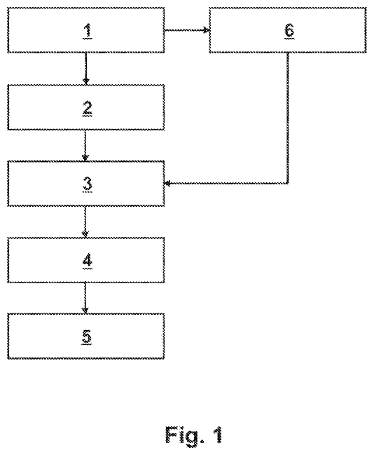

FIG. 1 shows a flow chart of a method for producing a sensor according to the invention;

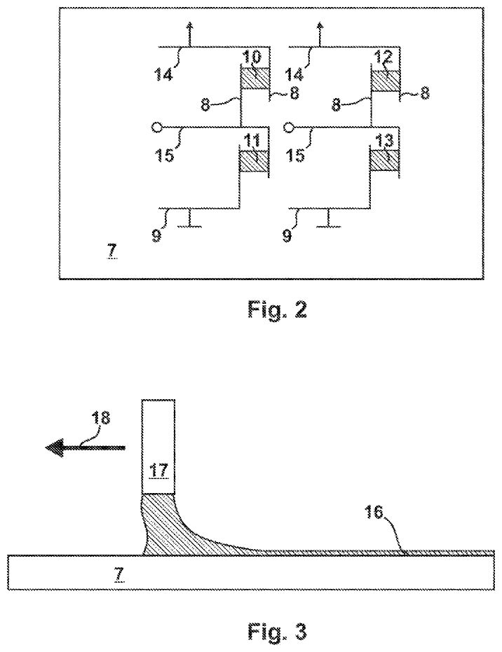

FIG. 2 shows a sensor according to the invention in schematic illustration;

FIG. 3 shows a process step during the application of a sensor substance;

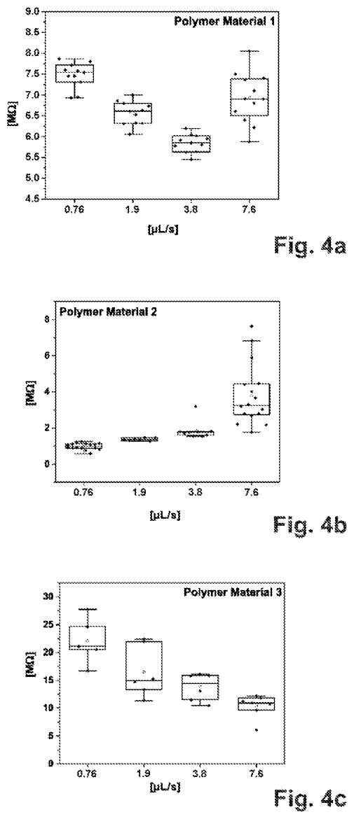

FIGS. 4a through 9c show relationships between parameters during an application of sensor substances and properties of the sensor substances;

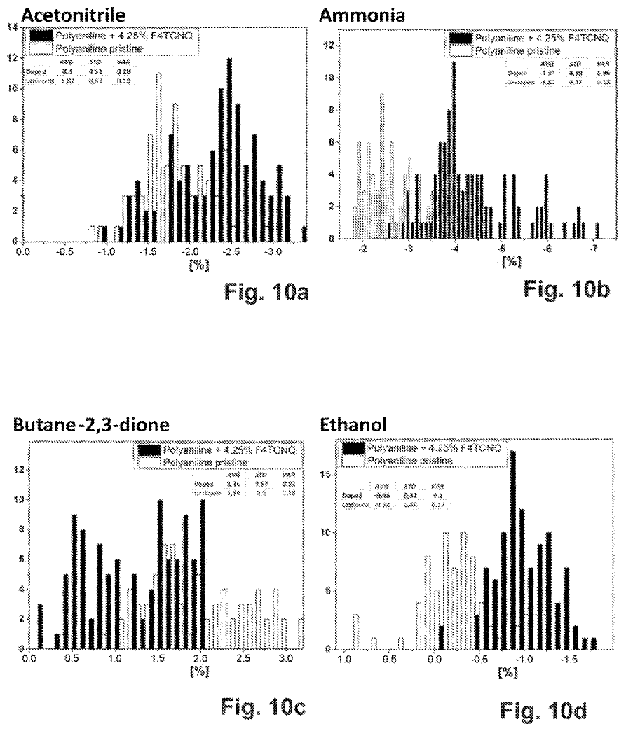

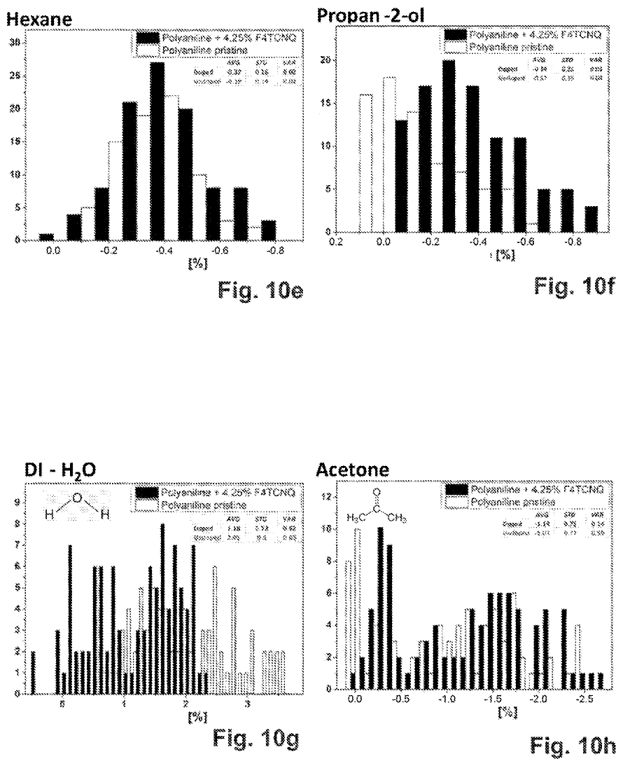

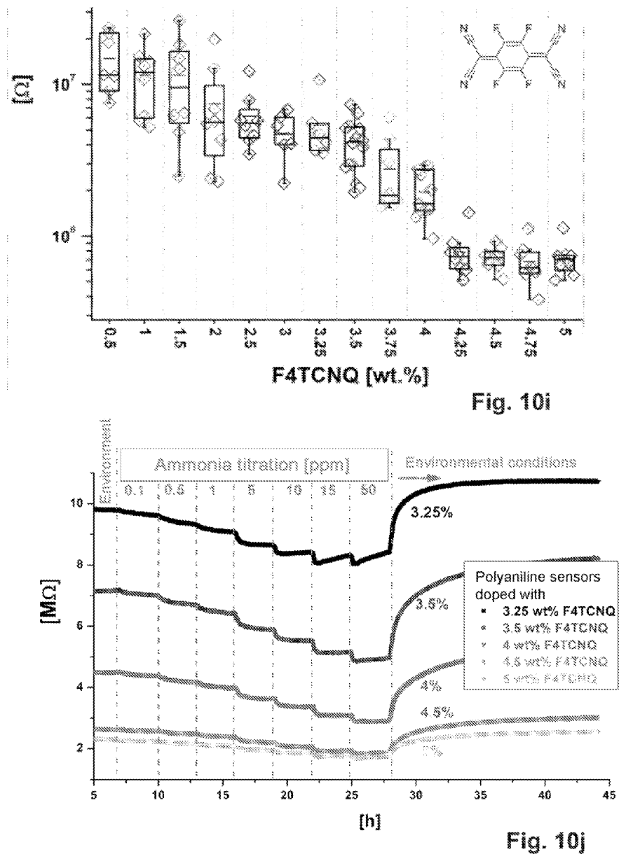

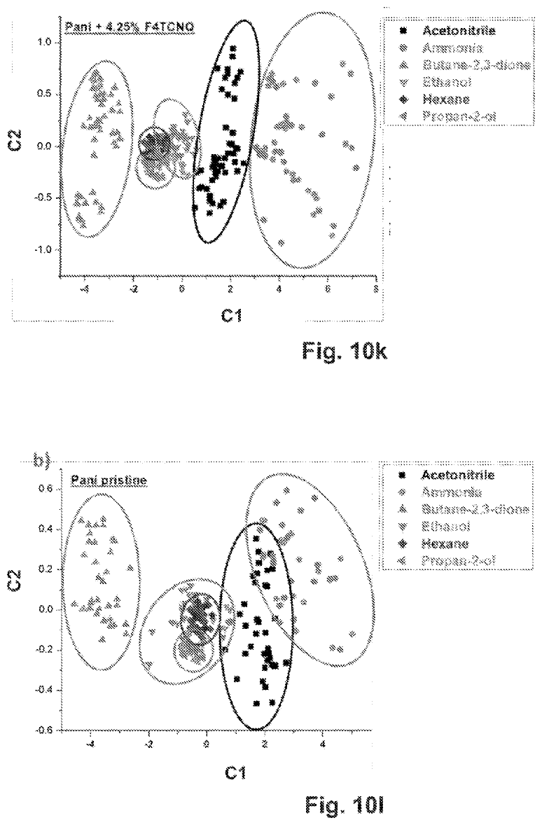

FIGS. 10a through 10h show relationships between a doping state of polyaniline and a behavior with respect to different gas constituents;

FIG. 10i shows effects of the doping level on a sensor resistance;

FIG. 10j shows effects of the doping level on the sensor resistance;

FIGS. 10k and 10l show effects of the doping level on the distinctness of different analytes.

FIG. 1 shows a flow chart of a method according to the invention. In a first step 1, a plurality of possible sensor substances 16 is thereby analyzed with respect to whether, and where applicable, how an electrical conductance of the respective sensor substance 16 changes when concentrations of different gases in an environment of the sensor substance 16 change. Tests are thereby preferably also carried out at different humidity levels of the air of an environment and different speeds at which the sensor substances 16 are applied in a liquid state to a substrate 7.

Results of said tests are stored in a database 6, so that, in the database 6, data with respect to a conductance of individual sensor substances 16 in the presence of different concentrations of different gases at different humidity levels of the air and different application speeds of the sensor substances 16, and possibly depending on other parameters, are available. The tests are typically carried out with different concentrations of volatile organic gases in an environment of the respective sensor substance 16. For example, conductances of different sensor substances 16 at different concentrations of the substances indicated in Table 3 in the air of an environment can be ascertained and stored in the database 6:

TABLE 3

| | | | | Trivial |
|---|---|---|---|---|
| Name | CAS | Formula | Assigned Smell | Name |
| Pentane-1,5- | 462- | C₅H₁₄N₂ | Decomposition | Cadaverine |
| diamine | 94-2 | | process | |
| Butane-1,4- | 110- | C₄H₁₂N₂ | Decomposition | Putrescine |
| diamine | 60-1 | | process | |
| Ethene | 74- | C₂H₄ | Ripening process | Ethene |
| | 85-1 | | of fruits | |
| Triethylamine | 121- | C₆H₁₅N | Fish | Triethylamine |
| | 44-8 | | | |
| Methylsulfonyl | 75- | C₂H₆S | Rotting process | Dimethyl |
| methane | 18-3 | | of vegetables | sulfide |
| 1-Methyl-4-(1- | 138- | C₁₀H₁₆ | Rinds of citrus | DL-limonene |
| methylethenyl)cy- | 86-3 | | fruits and clean- | |
| clohexene | | | ing products | |
| | | | (added fragrance) | |
| Methyl acetate | 79- | C₃H₆O₂ | Vegetable oils; | Methyl acetate |
| | 20-9 | | solvents, glue | |
| | 64- | C₂H₄O₂ | Sour, vinegar-like | Acetic acid |
| | 19-7 | | | |
| Ethanol | 64- | C₂H₆O | Fermentation | Ethanol |
| | 17-5 | | process, alcohol | |
| | | | residues | |
| Propan-2-ol | 67- | C₃H₈O | Cleaning agents, | Isopropanol |
| | 63-0 | | disinfectants | |
| Propan-2-one | 67- | C₂H₆O | Fermentation | Acetone |
| | 64-1 | | process, nail | |
| | | | polish, solvents | |

Based on this data, it is thus possible to produce sensors having the respective sensor substances 16 with which corresponding odors can be cost-efficiently ascertained, or with which concentrations of the gases that initiate corresponding olfactory perceptions in humans can be ascertained.

In a second step 2, requirements are defined for the sensor with respect to gases that are to be ascertained and usage conditions.

In a third step 3, at least two sensor substances 16 are selected based on the requirements and data stored in the database 6, which sensor substances 16 are subsequently integrated into a circuit in a fourth step 4, so that, by means of the circuit, conductances of the individual sensor substances 16 can be ascertained, in order to deduce concentrations of gases present in the environment via the conductances.

In an optional fifth step 5, the sensor is then calibrated in that the sensor is exposed to defined boundary conditions, in particular with respect to concentrations of specific gas constituents in an environment, whereupon conductances of the individual sensor substances 16 are measured via the circuit.

FIG. 2 shows a corresponding sensor in schematic illustration. As can be seen, the sensor comprises a circuit attached to a substrate 7, which is normally composed of an electrically non-conducting material, which circuit has electrodes or electrical conductors 8 that can, for example, be composed of copper or gold, wherein the electrodes are connected by sensor substances 16 in order to determine conductances of the individual sensor substances 16 via the electrodes. The sensor substances 16 are thereby printed onto the substrate 7, typically at a thickness of less than one millimeter and a predefined speed, since a speed at which the sensor substance 16 is printed onto the substrate 7 often also has an influence on a behavior of the sensor substance 16.

The illustrated sensor comprises two different sensor substances 16, wherein a first contact element formed by a sensor substance 16 that can, for example, be formed by one of the substances stated in Table 1 or one of the substances stated in Table 2 can be arranged between a voltage electrode 14, or an electrical conductor 8 at which a voltage of +1.5 V or +5 volts compared to a reference potential is present during operation of the sensor, and a measurement electrode 15, so that the measurement electrode 15 is essentially only electrically connected to the voltage electrode 14 by the first contact element 10.

The measurement electrode 15 is furthermore connected to a ground electrode 9 by a second contact element 11, at which ground electrode 9 the reference potential is present, wherein the second contact element 11 is likewise formed by the sensor substance 16 that also forms the first contact element 10. Thus, in combination with the measurement electrode 15, the voltage electrode 14, and the ground electrode 9, the first contact element 10 and the second contact element 11 form a voltage divider, so that, in principle, a voltage corresponding to half of the voltage of the voltage electrode 14 is present at the measurement electrode 15 if the conductances or resistances of the first contact element 10 and second contact element 11 are identical.

For an operation of the sensor, the first contact element 10 or the second contact element 11 is hermetically separated from an environment, for example by application of a coating to the first contact element 10. It is thus ensured that a change in a composition of the air of an environment only results in the change in a conductance of the second contact element 11, whereas the first contact element 10 remains as a reference in the circuit. As a result, measurement errors, for example, based on changes in the conductances of the corresponding sensor substance 16 that are independent of the composition of the air of an environment, in particular aging effects and temperature influences, can easily be avoided. Thus, with the first contact element 10 and the second contact element 11, a measured value that corresponds to a sensitivity of the first sensor substance 16 is obtained.

In the case of the illustrated sensor, a further measured value based on a third contact element 12 and a fourth contact element 13 can be analogously obtained, which third and fourth contact elements are respectively composed of a second sensor substance 16 that is different from the sensor substance employed in the first contact element 10 and second contact element 11, and can likewise be taken from Table 1 or Table 2. In this case, either the third contact element 12 or the fourth contact element 13 is once again hermetically separated from an environment.

The sensor substance 16 which forms the first contact element 10 and second contact element 11 can, for example, have a high sensitivity for a first gas constituent that is to be ascertained, and the sensor substance 16 which forms the third contact element 12 and fourth contact element 13 can have a high sensitivity or a selectivity for a second gas constituent that is to be ascertained, so that, using the sensor, the presence and, where applicable, the concentration of two different gas constituents in an environment of the sensor can be ascertained.

Of course, as an alternative to the illustrated circuitry, alternative circuits of the sensor substances 16 are also conceivable in order to ascertain the conductances of the sensor substances 16, for example a matrix circuit.

Depending on the requirements for the sensor, more than two different sensor substances 16 can also be arranged on the sensor. For example, a sensor according to the invention can comprise twelve different sensor substances 16 in 24 contact elements that are arranged on the sensor in a circuit similar to that illustrated in FIG. 2, in order to be able to measure changes in the conductances with twelve sensor substances 16.

In the application illustrated in FIG. 2, a change in the conductances acts as a potential shift at the measurement electrode 15. It is thus not the absolute conductance which is ascertained, but rather the relative change in the two conductances of the first contact element 10 and second contact element 11. The measurement of this voltage can, for example, take place using an analog-to-digital converter which, in the case of identical conductances of the first contact element 10 and second contact element 11, receives 50% of the voltage present at the voltage electrode 14 as an analog input signal and digitalizes said value in order to pass it along to the data processing device. In the case of a change in the conductance of the first contact element 10 relative to the conductance of the second contact element 11, this voltage thus also changes, and the change in voltage can be measured using the analog-to-digital converter and further processed in an automated manner.

More than twelve sensor substances 16 are, of course, also possible. For example, with a sensor according to the invention, 96 different sensor substances can also be provided.

Particularly if a high number of sensor substances are envisaged, it can also be provided that electrodes are arranged on the sensor in fractal patterns.

As a result, different concentrations of different gases in an environment and/or a concentration of one gas in an environment can be ascertained with particular accuracy. Thus, one sensor substance 16 can also only be suitable for ascertaining a concentration of a gas constituent within a specific range and, if said concentration range is exceeded, can enter into saturation, for example, so that a plurality of different sensor substances 16 may be necessary to ascertain a concentration of a gas over a large range.

Accordingly, ascertaining certain gas mixtures with acceptable accuracy may only be possible through a combination of specific sensor substances 16. For example, the conductance of a sensor substance can increase with an increasing concentration of two different gas constituents in the environment, whereas the conductance of another sensor substance can, among other things, decrease with an increasing concentration of one of these two gas constituents in an environment. By producing a sensor which comprises both sensor substances, it is thus possible to deduce with precision the presence and, where applicable, a concentration of a specific gas in the environment, even though no single sensor substance has a conductance specifically dependent solely on this one gas; rather, the conductances can also each be dependent on different gases in an environment. Thus, through the combination of different sensor substances, the sensor can be more specific than each individual sensor substance.

For this purpose, a method in accordance with an algorithm can be carried out in a data processing device to which the sensor is connected as part of a sensor system, and with which the conductances of the sensor substances are measured and evaluated, which algorithm comprises a corresponding correlation. The correlation of the conductances of the individual sensor substances with the concentrations of the respective gas constituents can, for example, be empirically determined ahead of time and stored in a data storage device in the sensor system. Alternatively or additionally, the sensor system can, of course, also be connected to a central database via a, possibly wireless, data connection, so that corresponding correlations can also still be changed after initial operation without needing to make changes to the sensor system itself.

To calibrate a corresponding sensor, a sensor of this type with a plurality of different sensor substances 16 can be exposed to different concentrations of different gases in an environment. On the basis of conductances that the individual sensor substances then exhibit in the presence of the individual gas constituents, it is possible to deduce the concentrations of the individual gas constituents in the environment with the aid of different sensitivities of the individual sensor substances. Here, it is also possible to resort to methods known from a field of pattern detection (pattern recognition), in order to obtain a sensor with a high specificity for certain gas constituents through the combination of different sensor substances.

For an operation, the sensor is typically integrated into a sensor system which comprises a data processing device and a data storage device, so that it is possible, for example, on the basis of calculated correlations that are determined in advance and stored in the data storage device and measured conductances, to be able to deduce specific gas constituents. A regression analysis can in particular also be employed for this purpose.

It is particularly preferred if the sensor can thereby be detachably connected to the data processing device, specifically by a plug connection. As a result, the most diverse sensors, which comprise different sensor substances 16 depending on the application case, can be combined with the same data processing device or the same type of data processing device, so that a highly identical part can be achieved with respect to the data processing device, even though the individual sensor systems are used for the most different applications.

FIG. 3 shows a process step of printing a sensor substance 16 onto a substrate 7 in a method according to the invention. As can be seen, the sensor substance 16 that has been brought into a liquid state is applied to an electrically non-conducting substrate 7 by means of a pneumatic nozzle, wherein the needle typically has a diameter of less than one millimeter. Inside the needle, the sensor substance 16 can thereby be under a negative pressure or vacuum and, for example, can be extruded out of the needle and brought into connection with the substrate 7 by only a brief pressure pulse or also by a continuous pressure, whereupon the needle is moved over the substrate 7 in order to apply the sensor substance 16 to the substrate 7. The needle is thereby normally moved over the substrate 7 at a speed of 0.2 mm/s to 10 mm/s along a feed direction 18, so that the sensor substance 16 remains on the substrate 7 with a layer thickness of 0.5 mm, for example, in a hardened state.

In principle, the speed can also be 0 mm/s. The application is then equivalent to pipetting, but can also be controlled via the pressure at which the sensor substance 16 is applied to the substrate 7. Furthermore, alternatively or additionally, it is also possible to pipette directly, wherein an application of the sensor substance 16 to the substrate 7 can be controlled via a respectively applied volume.

Typically, the sensor substance 16 is brought into a liquid state by a solvent. In order to ensure reproducible material properties of the sensor substance 16 on the substrate 7 during printing, the sensor substance 16 is preferably applied to the substrate 7 when the substrate 7 is kept at a predefined temperature. For this purpose, the substrate 7 can, for example, be arranged on a hot plate not illustrated in FIG. 3, wherein a temperature is typically higher than a temperature at which the solvent that was used to bring the sensor substance 16 into a liquid state vaporizes.

A corresponding sensor can in particular comprise the substances stated in Table 2, in order to be able to ascertain the gases in an environment that trigger in a human the olfactory sensations indicated in the last column in Table 2. In particular, a sensor of this type can also be employed to ascertain contents of a refuse container on the basis of a gas composition, especially since an identification of volatile organic gases in particular such as those which appear during the decomposition of waste materials in a refuse container, for example, can be ascertained with particular accuracy using these sensor substances 16.

FIGS. 4a through 4c show a relationship between a speed of an application of a sensor substance formed by a polymer and an electrical resistance of said sensor substance on the finished sensor for four different sensor substances, for example one of the sensor substances noted in Table 1 or Table 2, which are labeled as Polymer Material 1, Polymer Material 2, and Polymer Material 3. The charts were created using measurements, so that, on the basis of multiple measuring points, statistically relevant data such as a variance and a median can also be seen. Other parameters that are relevant for printing are typically not changed during the creation of these charts, so as to be able to assess the influence of the printing speed in an isolated manner.

Of course, series of measurements can also be performed to create corresponding charts, in which series initially only one parameter, such as the printing speed, is changed, and said series of measurements is carried out for different temperatures in order to be able to assess potential interactions.

As can easily be recognized with the aid of the box plot illustration, the speed of application depicted on the horizontal axis, which can also be referred to as the printing speed or deposition rate, has an influence on the resistance of the sensor substance on the sensor, which resistance is depicted on the vertical axis. Typically, a lower resistance is more beneficial, so that for the relationship of the electrical resistance and the printing speed illustrated in FIG. 4a for a first sensor substance or Polymer Material 1, a printing speed of 3.8 μL/s is thus beneficial.

With the sensor substance depicted in FIG. 4b, the variance and median of the resistance increase markedly at a higher printing speed, so that, in this case, a printing speed of less than 3.8 μL/s is beneficial to obtaining a low resistance, for example a speed of 0.76 μL/s to 1.9 μL/s.

The relationship of the resistance of Polymer Material 3 to the printing speed depicted in FIG. 4c is reversed. In this case, both the median and also a variance of the resistance decrease with an increasing printing speed. The result is that different printing speeds are beneficial for different sensor substances, in order to be able to obtain a desired sensor behavior not only through the selection of the sensor substances, but additionally also through the selection of the parameters for the application.

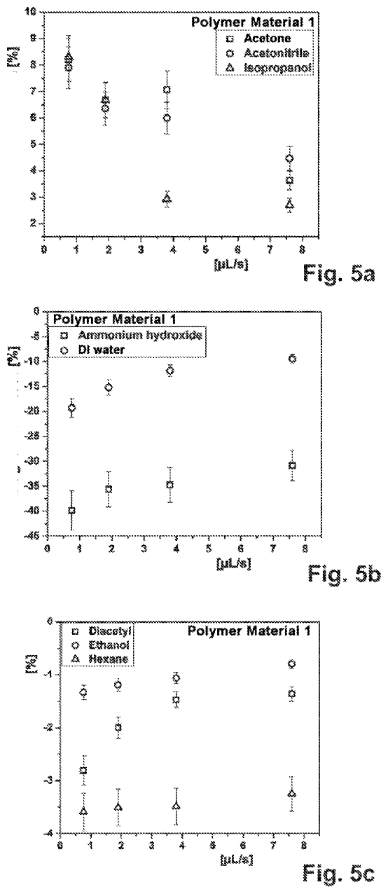

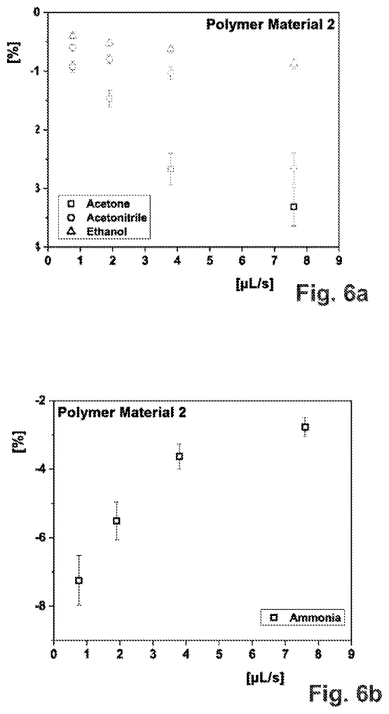

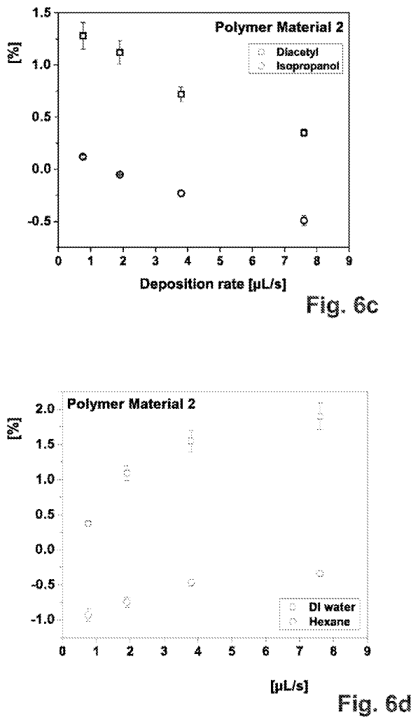

FIGS. 5a through 6d show relationships between the printing speed depicted on the horizontal axis on the one hand and a sensor performance depicted on the vertical axis. The sensor performance is thereby represented by a change in the sensor signal due to the exposure to the respective gas constituent, which change is indicated in relative terms, namely using the value of the signal after an exposure time of 300 seconds in relation to the value of the signal prior to exposure of the sensor substance to the respective gas constituent. Higher values thus indicate a better sensor performance with respect to the respective gas constituent, since the sensor substance in that case exhibits a higher sensitivity for the respective gas constituent, that is, the signal changes more markedly when the sensor substance is exposed.

FIGS. 5a through 5c show this relationship with respect to a sensor substance referred to as Polymer Material 1, which can, for example, correspond to one of the sensor substances noted in Table 1 or Table 2. As follows from FIG. 5a, lower printing speeds result in greater changes in the sensor signal, for example, in a resistance of the sensor substance or a voltage drop across the sensor substance, with respect to acetone, acetonitrile, and isopropanol. Accordingly, a low printing speed of 1 μL/s, for example, is in this case beneficial for the sensor performance of Polymer Material 1 with respect to acetone, acetonitrile, and isopropanol.

FIG. 5b shows the behavior with respect to ammonium hydroxide and deionized water.

As can be seen from FIG. 5c, changes in the sensor signal with respect to ethanol and hexane are not dependent on the printing speed to an equally pronounced extent, but a behavior dependent on the printing speed does also result in this case.

FIGS. 6a through 6d show corresponding charts for a different sensor substance, which is referred to here as Polymer Material 2, and can, for example, also be formed by one of the substances noted in Tables 1 and 2. As can be seen here, with this sensor substance a higher printing speed or deposition rate of 7 μL/s, for example, is beneficial for a marked reaction of the sensor substance with respect to acetone, acetonitrile, and ethanol, whereas a higher speed is beneficial for the detection of ammonia.

By utilizing these effects, a sensor having multiple identical sensor substances can be produced, for example, which sensor substances are, however, applied at different printing speeds, so that with the Polymer Material 2 sensor substance applied at low printing speed, acetone, acetonitrile, and ethanol can be detected well, whereas the same sensor substance applied at a high printing speed is beneficial for the identification of ammonia.

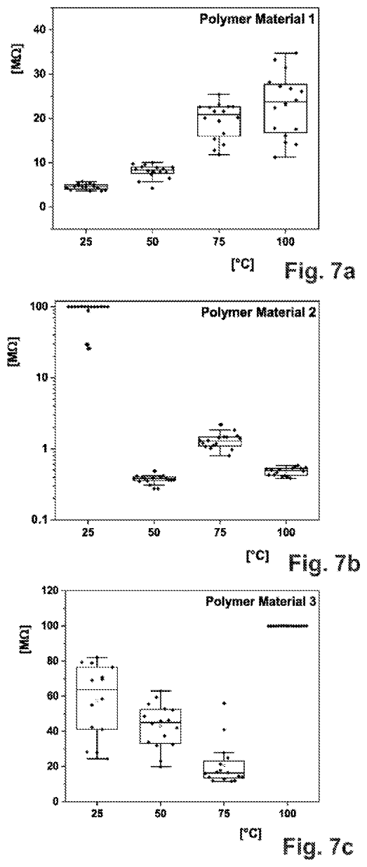

FIGS. 7a through 9d show an influence of the temperature during the application of the sensor substance to the substrate or during printing.

FIGS. 7a through 7c thereby show effects of the temperature on the resistance of the sensor substance for three different polymers. As can be seen in FIG. 7a, the resistance in the case of Polymer Material 1 increases with increasing temperature, whereas the resistance in the case of Polymer Material 2 increases up to a temperature of 75° C. and then decreases again as the temperature increases, as is depicted in FIG. 7b.

FIG. 7c shows a relationship opposite of that depicted in FIG. 7a, wherein the resistance decreases with an increasing printing speed.

Through the combination of parameters during the application of the sensor substance, for example with respect to the speed and the temperature, it is thus of course possible to achieve a correspondingly pronounced effect on the behavior of the sensor substance, in order to obtain, for example, a sensor with a particularly low resistance, which sensor is sensitive to one or more specific gas constituents.

Of course, other parameters can also be relevant for a sensor behavior, for example a nozzle cross-section of a nozzle from which the liquid sensor substance is applied during printing.

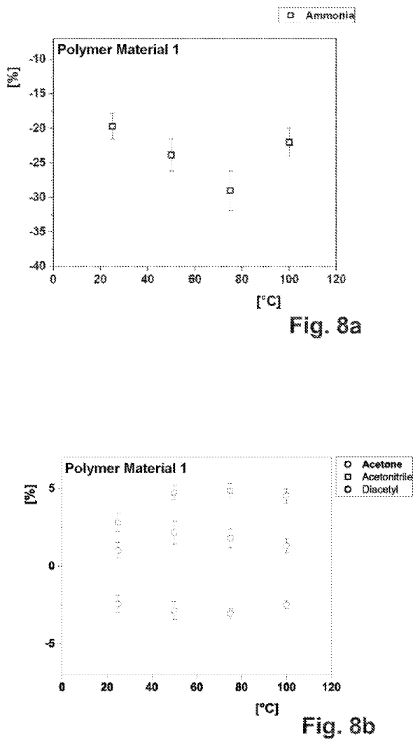

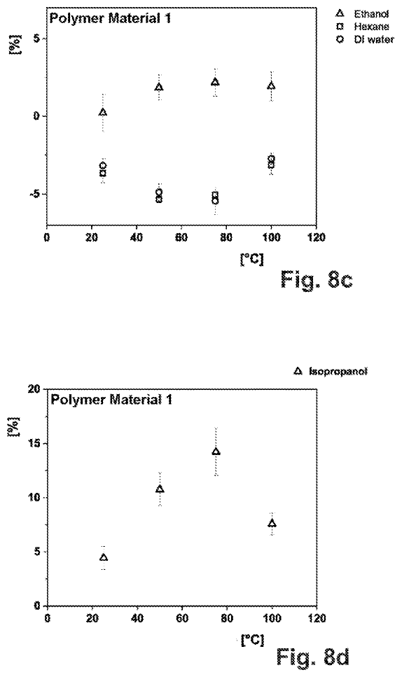

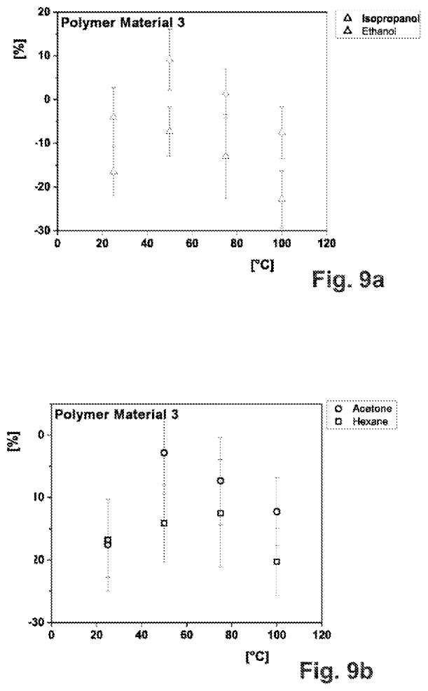

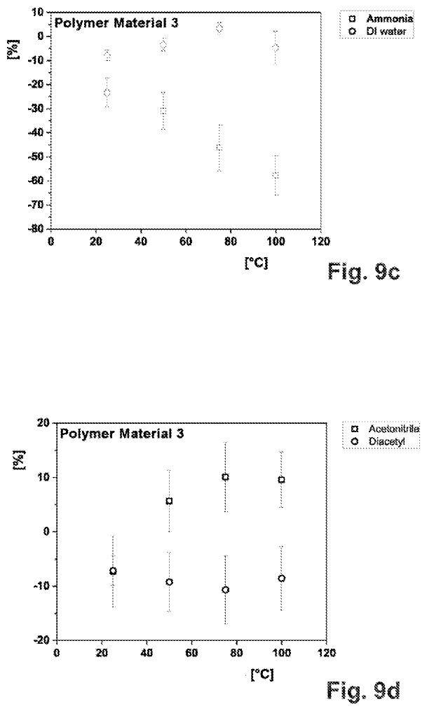

FIGS. 8a through 9d show the behavior of different sensor substances when exposed to different gas constituents, depending on the temperature during printing.

Here, the temperature is depicted on the horizontal axis and the change in a signal of the sensor substance on the vertical axis in each case, that is, once again a resistance of the sensor substance after an exposure time of 300 seconds relative to the same signal prior to the beginning of exposure to the gas constituent, for example. High values thus indicate a relatively marked change in the signal or the conductance of the sensor substance in the presence of the respective gas constituent.

FIGS. 8a through 8d thereby show a change in a behavior of a first polymer, which is referred to as Polymer Material 1 and can be formed by one of the substances noted in Tables 1 and 2, for example. As can be seen, with the behaviors depicted in FIGS. 8a through 8d, high temperatures of approximately 80° C. during printing or during the drying of the sensor substance are beneficial to achieving a pronounced response of the sensor material, that is, a marked signal change, in the presence of ammonia, hexane, water, and isopropanol, whereas the temperature during printing has only a slight influence on the sensor performance in relation to acetone, acetonitrile, and diacetyl.

Similar trends are recognizable with respect to the behavior of Polymer Material 3 depicted in FIGS. 9a through 9d, which can likewise be formed by one of the substances noted in Tables 1 and 2.

FIGS. 10a through 10h show differences in the behavior of the sensor substance PANI, or polyaniline, between a pristine, or undoped, state and a state doped with 4.25% F4TCNQ. In each case, a change in the measurement result or a change in the resistance of the sensor substance due to an exposure of the sensor substance to the corresponding gas constituent in relation to the resistance prior to exposure is thereby plotted on the abscissa axis. On the ordinate axis, the number of corresponding measurement results is plotted. Correspondingly, according to FIG. 10a, the resistance of the polyaniline doped at 4.25 percent by weight when exposed to acetonitrile changed by negative 2.5 percent in twelve samples, whereas in the case of undoped polyaniline, a change by 2.5 percent only resulted from one sample. On average, the resistance in the case of doped polyaniline changes by negative 2.3 percent when exposed to acetonitrile, whereas the resistance in the case of undoped polyaniline changes on average by only negative 1.57 percent.

As can be seen, the different doping state has a more pronounced effect on a behavior with respect to 2,3-butanedione and ethanol than on a behavior with respect to hexane. It is thus also possible, through a simple doping of sensor substances, to influence a behavior in a targeted manner, for which reason it is beneficial to also include the doping state when creating a database which contains behaviors of different sensor substances with respect to different gas constituents. In this manner, an appropriately doped sensor substance can be selected when producing the sensor. To create the figures, two sensors were produced which each contained multiple sensor substances, the conductances of which were measured, wherein a first sensor respectively contained undoped sensor substances and a second sensor respectively contained sensor substances doped with 4.25% F4TCNQ, and said sensors were each exposed for 300 seconds to identical environment conditions with respect to the gas constituents stated in the figures. The undoped sensor substances, each undoped polyaniline in this case, are thereby represented by white bars, and the doped sensor substances, each polyaniline doped with 4.25% F4TCNQ in this case, are represented by black bars in the figures. As can be seen, the measured values are also dispersed within the undoped sensor substances and within the doped sensor substances, but the average of the individual measured values changes with respect to some gas constituents due to the doping, so that a sensor behavior of individual sensor substances with respect to specific gas constituents can be influenced in a targeted manner by doping.

FIG. 10i shows effects of the doping level on the sensor resistance. The doping level of F4TCNQ in percent by weight is thereby depicted on the abscissa axis, and measurement results of a resistance of the sensor substance, for example polyaniline, are depicted in ohms on the ordinate axis, so that the extent to which the results are dispersed is also evident. As can be seen, a doping amount of 4.25 percent by weight F4TCNQ results in a beneficial behavior of reproducibility and conductivity.

FIG. 10j shows effects of the doping level on the sensor resistance or the sensitivity at different concentrations of ammonia. An exposure time is depicted in hours on the abscissa axis, whereas a resistance of the sensor substance, for example polyaniline, is depicted in megaohms on the ordinate axis. As can be seen with the aid of the dashed lines, measurement began with ambient air, whereupon the concentration of ammonia was initially increased to 0.1 ppm and, at 25 hours, ultimately to 50 ppm, after which the sensor substances were once again exposed to the ambient air without ammonia. The resistance of the sensor substance thereby generally decreases with an increasing doping level, though the sensitivity also decreases. Here, a doping level of 4.25 percent by weight F4TCNQ is also a good compromise between low resistance and good sensitivity.

FIGS. 10k and 10l show effects of the doping level of polyaniline on the distinctness of different analytes. The figures thereby show principal components which were ascertained in the course of a principal component analysis.

The principal components thereby result following an extraction of information from sensor responses for all polymers in the integrated circuit with n sensor elements. The extracted information, in this case the sensor response after 600 seconds, an exponential fit, and the slope of the curve, is then plotted in an n-dimensional vector space and, by means of principal component analysis, that vector orthogonal to a plane, that is, a viewing angle, is sought at which the best distinctness of the data points can be seen. This is also referred to as principal axis transformation.

The two axes of the plane thus found are referred to as primary components and are also depicted in the abscissa axis and ordinate axis in FIGS. 10k and 10l. Denoted as “Hauptkomponenten” in German, these can also be referred to as principal components, for which reason the axes in the charts are labeled C1 and C2.

FIG. 10k thereby shows a behavior of polyaniline doped with 4.25 percent by weight F4TCNQ and FIG. 10j a behavior of undoped polyaniline when exposed to different analytes. As follows from these figures, doping improves the distinctness. Thus, with undoped polyaniline, principal component analysis yields similar values for exposure to ethanol, hexane, and propane-2-ol, whereas polyaniline doped with 4.25% percent by weight F4TCNQ has better-distinguishable principal components that are dependent on the three analytes noted.

A sensor according to the invention can be produced at very low cost, so that a sensor of this type can be employed for the most diverse applications, for example in order to continuously ascertain, in refuse containers, a composition of waste located in the refuse container, which composition can in turn be transmitted to a waste disposal entity via a modem, for example, so that, at an appropriate degree of fullness of the refuse container, the collection vehicle that corresponds to the respective waste can be dispatched to the refuse container.

1. A method for producing a sensor comprising a circuit with multiple sensor substances, wherein the electrical conductivities of sensor substances from a sensor substance list, which sensor substances each have an electrical conductivity dependent on a concentration of one or more gases in an environment of the sensor substance, are measured in environments with different concentrations of different gases and optionally different humidity levels and/or different temperatures, in order to determine sensitivities of the conductivities of the individual sensor substances for the different gases, wherein the measured conductivities and determined sensitivities of the sensor substances from the sensor substance list are stored in a database, whereupon, depending on requirements for the sensor with respect to gas concentrations in an environment which are to be ascertained using the sensor as well as on predefined usage conditions, in particular with respect to a humidity level and/or a temperature, at least two sensor substances from the sensor substance list are selected for the gas concentrations that are to be ascertained, based on the sensitivity stored in the database for the respective sensor substances, which selected sensor substances are integrated into an electrical circuit such that a conductivity of the sensor substances can be measured, so that the concentration of the gas constituent that is to be ascertained and/or a change in the concentration of the gas constituent that is to be ascertained in the environment of the circuit can be ascertained by measurement of the conductances of the sensor substances in the circuit and/or by measurement of changes in the conductances of the sensor substances in the circuit wherein the sensor substances are applied in a liquid state to a substrate that comprises electrical conductors, whereupon the sensor substances harden on the substrate, wherein the sensor substances connect electrical conductors so that an electrical conductance of the sensor substances can be ascertained via the electrical conductors of the substrate, wherein sensitivities of the conductances of the sensor substances for different gases in an environment are ascertained depending on one or more printing parameters with which the sensor substances are applied to the substrate, whereupon these dependencies are stored in the database, after which, based on data stored in the database and depending on requirements for the sensor with respect to gas concentrations in an environment that are to be ascertained using the sensor as well as on predefined usage conditions, in particular with respect to a humidity level and/or a temperature, printing parameters for the application of the individual sensor substances to the substrate are selected, whereupon the sensor substances are applied to the substrate using corresponding printing parameters.

2. The method according to claim 1, wherein the conductivities of the sensor substances are measured at different gas concentrations, different humidity levels, and different temperatures.

3. The method according to claim 1, wherein the conductivities of the sensor substances are ascertained at different concentrations of one or more volatile organic gases in the atmosphere.

4. The method according to claim 1, wherein conductivities of at least one sensor substance are measured in different doping states, wherein a doped state of the sensor substance is formed, for example, by mixtures of the sensor substance, in particular a polymer, with a dopant, in particular F4TCNQ (2,3,5,6-tetrafluoro-7,7,8,8-tetracyanoquinodimethane).

5. The method according to claim 1, wherein the sensor substances are applied in a liquid state to a substrate, in particular to a printed circuit board, that comprises electrical conductors, whereupon the sensor substances harden on the substrate, wherein the sensor substances connect electrical conductors so that an electrical conductance of the sensor substances can be ascertained via the electrical conductors of the substrate.

6. The method according to claim 5, wherein the sensor substances are dissolved in a solvent in order to bring the sensor substances into a liquid state.

7. (canceled)

8. The method according to claim 5, wherein sensitivities of the conductances of the sensor substances for different gases in an environment are ascertained depending on a speed at which the sensor substances are applied to a substrate, whereupon these dependencies are stored in the database, after which, based on data stored in the database and depending on requirements for the sensor with respect to gas concentrations in an environment that are to be ascertained using the sensor as well as on predefined usage conditions, in particular with respect to a humidity level and/or a temperature, speeds for the application of the individual sensor substances to the substrate are selected, in particular speeds of 0.2 μL/s to 30 μL/s, whereupon the sensor substances are applied to the substrate at corresponding speeds.

9. The method according to claim 5, wherein the liquid sensor substances are applied to the substrate using a nozzle that is moved relative to the substrate, wherein the nozzle preferably has a nozzle diameter of less than 1 mm, in particular 100 μm to 500 μm.

10. The method according to claim 5, wherein the substrate is kept at a predefined temperature during the application of the sensor substances, in particular a temperature of 25° C. to 120° C.

11. The method according to claim 5, wherein the substrate is positioned on a heating device, in particular a hot plate, which is kept at a constant temperature, in particular a temperature at which a solvent with which the sensor substance is brought into a liquid state vaporizes.

12. The method according to claim 1, wherein at least one of the sensor substances that are integrated into the circuit comprises a polymer.

13. The method according to claim 1, wherein the sensor substances comprise metal oxides, in particular copper oxide or zinc oxide, metal particles, in particular gold particles, and/or polymers, preferably electrically conductive, in particular organic electrically conductive polymers, or are composed thereof.

14. The method according to claim 1, wherein, following integration of the sensor substances into the circuit, the sensor is calibrated in that the conductances of the individual sensor substances of the sensor are measured depending on the individual gas concentrations in an environment that are to be ascertained, the gas concentrations present in the environment during the calibration as well as a temperature and a relative humidity of the air being known, wherein the conductances of the sensor substances of the sensor are stored.

15. The method according to claim 1, wherein the conductances of the individual sensor substances of the sensor are measured at different temperatures, different humidity levels, and different exposure times, in order to ascertain influences of moisture and temperature on a sensor behavior.

16. The method according to claim 1, wherein the conductances of the individual sensor substances of the sensor are ascertained in the presence of multiple different gases in an environment of the sensor.

17. A sensor which is suitable for measuring one or more specific gas constituents in the air of an environment under predefined usage conditions, in particular with respect to a humidity level and/or a temperature, comprising an electrical circuit, wherein the sensor comprises at least two sensor substances from a sensor substance list, wherein conductivities of the sensor substances can be ascertained by the circuit, wherein the sensor is in particular produced in a method according to claim 1.

18. The sensor according to claim 17, wherein at least six, preferably at least twelve, different sensor substances are arranged on the circuit, wherein the conductivity of each of said sensor substances can be ascertained.

19. The sensor according to claim 17, wherein the sensor substance list contains polymers, preferably conductive, in particular organic electrically conductive polymers, and/or metal oxides, in particular copper oxide or zinc oxide, and/or metal particles, in particular gold particles, or is composed of substances of this type.

20. The sensor according to claim 17, wherein the sensor substances are printed onto the circuit.

21. The sensor according to claim 17, wherein electrodes that are connected by the sensor substances are arranged on the circuit.

22. The sensor according to claim 17, wherein, in parallel and/or in series with each sensor substance that connects two electrodes of the circuit and is connected to an environment, an identical sensor substance is arranged in the circuit, which identical sensor substance is hermetically separated from the environment.

23. A sensor system having a sensor and a data processing device connected to the sensor, which data processing device in particular comprises a microprocessor and a data storage device, wherein the sensor is embodied according to claim 17.

24. The sensor system according to claim 23, wherein the sensor can be detachably connected to the data processing device.

25. The sensor system according to claim 23, wherein the sensor is embodied as a plug-in card and is connected to the data processing device via a slot, so that the sensor can be plugged into the slot, wherein an electrical connection between the sensor and the data processing device can be produced by plugging the sensor into the slot.

26. The sensor system according to claim 23, wherein the data processing device is suitable for performing a method wherein, with the aid of measured conductances of the sensor substances as well as sensitivities stored in a database for the individual sensor substances, concentrations of one or more gases in an environment of the sensor are ascertained.

27. The sensor system according to claim 23, wherein the sensor system comprises an acoustic sensor and/or an optical sensor, in order to perform a measurement by the sensor, in particular such that said measurement is triggered by acoustic and/or optical changes in an environment of the sensor system.

28. The sensor system according to claim 23, wherein a transport device is provided with which a gas that is to be analyzed can be actively transported to the sensor substances of the sensor, in particular a pump, a compressor, a blower, or the like.

29. A method for ascertaining one or more specific gas constituents in an environment under predefined usage conditions, in particular with respect to a humidity and/or a temperature, using a sensor system, wherein a sensor system according to claim 23 is employed.

30. The method according to claim 29, wherein, cross-correlations of conductances of the sensor substances of the sensor's sensor substances are taken into consideration when ascertaining the gas constituents.

31. The method according to claim 29, wherein, with the aid of measured conductances of the sensor substances and sensitivities stored in a database for the individual sensor substances, concentrations of one or more gases in an environment of the sensor are ascertained.

32. The method according to claim 29, wherein the method is employed to ascertain substances contained in a refuse container

33. The method according to claim 29, wherein a trigger event is captured and an analysis of gas constituents is carried out starting with the trigger event.

34. The method according to claim 29, wherein, on the basis of chronological changes in the conductances of the individual sensor substances that are measured starting with the trigger event, concentrations and/or changes of gas constituents in an environment of the sensor are ascertained.
